SEQUENCE LISTING (2 sequences)

SEQ ID NO: 1 - protein, 233 aa, Porcine circovirus
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 2 - protein, 233 aa, Porcine circovirus
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

(19) United States
(12) Patent Application Publication
(10) Pub. No.: US 2014/0322267 A1
(43) Pub. Date: Oct. 30, 2014

(54) ORF2 PROTEIN OF PCV2 SUBTYPE A (PCV2A) FOR USE IN CROSS-PROTECTION

(71) Applicants: Gregory HAIWICK, Ankeny, IA (US); Luis HERNANDEZ, Story City, IA (US); Wesley Scott JOHNSON, Ames, IA (US); Christine Margaret MUEHLENTHALER, Ames, IA (US); Eric Martin VAUGHN, Ames, IA (US)

(72) Inventors: Gregory HAIWICK, Ankeny, IA (US); Luis HERNANDEZ, Story City, IA (US); Wesley Scott JOHNSON, Ames, IA (US); Christine Margaret MUEHLENTHALER, Ames, IA (US); Eric Martin VAUGHN, Ames, IA (US)

(73) Assignee: BOEHRINGER INGELHEIM VETMEDICA, INC., St. Joseph, MO (US)

(21) Appl. No.: 14/263,193

(22) Filed: Apr. 28, 2014

Related U.S. Application Data
(60) Provisional application No. 61/817,658, filed on Apr. 30, 2013.

(51) Int. Cl. A61K 39/12; A61K 39/108; A61K 39/08; A61K 39/02; A61K 39/215; A61K 39/145; A61K 39/04; A61K 39/085; A61K 39/09; A61K 39/23; A61K 39/245; A61K 39/05; A61K 39/235

(52) U.S. Cl. CPC A61K 39/12; A61K 39/05; A61K 39/0258; A61K 39/08; A61K 39/0208; A61K 39/215; A61K 39/235; A61K 39/04; A61K 39/085; A61K 39/092; A61K 39/23; A61K 39/245; A61K 39/145; A61K 2039/70
USPC 424/201.1; 424/204.1; 424/202.1

(57) ABSTRACT

Vaccination methods to control PCV2 infection with different PCV2 subtypes are disclosed. Specifically, a PCV2 subtype a (PCV2a) ORF2 proteins or immunogenic compositions comprising a PCV2a ORF2 protein are used in a method for the treatment or prevention of an infection with PCV2 of a different subtype, the reduction, prevention or treatment of clinical signs caused by an infection with PCV2 of a different subtype, or the prevention or treatment of a disease caused by an infection with PCV2 of a different subtype.

### SEQUENCE LISTING

This application contains a sequence listing in accordance with 37 C.F.R. 1.821-1.825. The sequence listing accompanying this application is hereby incorporated by reference in its entirety.

### BACKGROUND OF THE INVENTION

Porcine circovirus type 2 (PCV2) is a small (17-22 nm in diameter), icosahedral, non-enveloped DNA virus, which contains a single-stranded circular genome. PCV2 shares approximately 80% sequence identity with porcine circovirus type 1 (PCV-1). However, in contrast with PCV1, which is generally non-virulent, swine infected with PCV2 exhibit a syndrome commonly referred to as Post-weaning Multisystemic Wasting Syndrome (PMWS). PMWS is clinically characterized by wasting, paleness of the skin, unthriftiness, respiratory distress, diarrhea, icterus, and jaundice. In some affected swine, a combination of all symptoms will be apparent while other swine will only have one or two of these symptoms. During necropsy, microscopic and macroscopic lesions also appear on multiple tissues and organs, with lymphoid organs being the most common site for lesions. A strong correlation has been observed between the amount of PCV2 nucleic acid or antigen and the severity of microscopic lymphoid lesions. Mortality rates for swine infected with PCV2 can approach 80%. In addition to PMWS, PCV2 has been associated with several other infections including pseudorabies, porcine reproductive and respiratory syndrome (PRRS), Glasser's disease, streptococcal meningitis, salmonellosis, postweaning colibacillosis, dietetic hepatosis, and suppurative bronchopneumonia.

Currently, there are three subtypes of PCV2 known (PCV2a, PCV2b and PCV2c), which are classified according to a unified nomenclature for PCV2 genotypes (Segales, J. et al., 2008, PCV-2 genotype definition and nomenclature, Vet Rec 162:867-8). Two further subtypes (PCV2d and PCV2e) have been proposed (Wang et al. Virus Res. 2009 145(1):151-6) but it was demonstrated later that they belong to the PCV2a and PCV2b clusters (Cortey et al. Vet Microbiol. 2011 149(3-4):522-32011). According to this unified nomenclature for PCV2 genotypes the ORF2 gene is used to perform genotyping for PCV-2, wherein the geotyping is based on the proportion of nucleotide sites at which two sequences being compared are different (p distance). This value is obtained by dividing the number of nucleotide differences by the total number of nucleotides compared (Kumar et al. 2001 Bioinformatics 17, 1244-1245) and subsequently, the construction of a p distance/frequency histogram enables to determine potential cut-off values to distinguish different genotypes (Rogers and Harpending 1992 Molecular Biology and Evolution 9, 552-569; Biagini et al. 1999 Journal of General Virology 80, 419-424). Using this methodology, ORF2 PCV-2 sequences are assigned to different genotypes when the genetic distance between them is 0-035.

Fort et al. (Vaccine 2008 26(8):1063-71) discloses a prime and boost vaccination with a PCV2 subunit vaccine containing the capsid protein of a genotype 2 strain of PCV2 in an oil-in-water emulsion, wherein piglets were vaccinated at 4 weeks of age and revaccinated two weeks later. Four different PCV2 isolates obtained from lymphoid tissues of PMWS-affected pigs were used as challenge viruses according to Fort et al., wherein two of the strains were classified into genotype 1 and two into genotype 2. Fort et al. further describes that the experimental model used in this study did not succeed in causing PMWS. It was, however, shown that the vaccine, administered in a prime and boost vaccination regimen, was able to prevent viremia in all vaccinated animals regardless of the virus used for the challenge. In contrast, nasal and faecal shedding was not fully prevented according to Fort et al., although in both cases the percentage of positive pigs and the viral load in nasal cavities and faecal swabs were significantly reduced in the two shot vaccinated pigs compared to their non-vaccinated counterparts.

Thus, there is a need of new and enhanced vaccination approaches to control PCV2 infection with different PCV2 subtypes.

### SUMMARY OF THE INVENTION

The solution to the above technical problem is achieved by the description and the embodiments characterized in the claims.

Thus, the invention in its different aspects is implemented according to the claims.

The invention is based on the surprising finding that the administration of only one dose of PCV2 subtype a (PCV2a) ORF2 protein, in particular if it is contained in an immunogenic composition, wherein preferably the virucidal activity of the immunogenic composition has been reduced, is sufficient for reducing and preventing clinical signs caused by an infection with PCV2 of a subtype other than subtype 2a.

In one aspect, the invention thus relates to PCV2 subtype a (PCV2a) ORF2 protein or an immunogenic composition comprising PCV2a ORF2 protein for use in a method for the treatment or prevention of an infection with PCV2 of a different subtype, the reduction, prevention or treatment of clinical signs caused by an infection with PCV2 of a different subtype, or the prevention or treatment of a disease caused by an infection with PCV2 of a different subtype.

### DETAILED DESCRIPTION OF THE INVENTION

It is hence understood that the term “PCV2 of a different subtype”, as mentioned herein, relates to PCV2 of a subtype other than subtype 2a.

As used herein, the terms “PCV2 of a subtype other than subtype 2a” and “PCV2 of a different subtype” are thus equivalent.

The term “PCV2 subtype a (PCV2a) ORF2 protein”, as described herein, relates to the protein encoded by the ORF2 gene of a PCV-2a as defined by the standardized nomenclature for PCV2 genotype definition (Segales, J. et al., 2008, PCV-2 genotype definition and nomenclature, Vet Rec 162:867-8, which is incorporated herein by reference).

Preferably, the infection with PCV2 of a different subtype or with PCV2 of a subtype other than subtype 2a, respectively, is an infection with PCV2 subtype b (PCV2b) and/or PCV2 subtype c (PCV2c).

More preferably, the infection with PCV2 of a different subtype or with PCV2 of a subtype other than subtype 2a, respectively, is an infection with PCV2b.

According to a particular preferred aspect, the infection with PCV2 of a different subtype or with PCV2 of a subtype other than subtype 2a, respectively, is a concurrent infection with (i) PCV2 of a different subtype or with PCV2 of a subtype other than subtype 2a, respectively, and (ii) PCV2a.

According to a more particular embodiment, the infection with PCV2 of a different subtype or with PCV2 of a subtype other than subtype 2a, respectively, is a concurrent infection with PCV2a and PCV2b.

The terms “PCV2a”, “PCV2b” and “PCV2c”, respectively, as described herein, relate to PCV-2a, PCV-2b and PCV-2c, respectively, according to the standardized nomenclature for PCV2 genotype definition (Segales, J. et al., 2008, PCV-2 genotype definition and nomenclature, Vet Rec 162:867-8, which is incorporated herein by reference).

In particular, said concurrent infection with PCV2a is an infection with (i) a PCV2 comprising a polypeptide that is at least 94%, preferably at least 95%, more preferably at least 96%, still more preferably at least 97%, yet more preferably at least 98%, and most preferably at least 99% identical to the sequence of SEQ ID NO: 1 or (ii) a PCV2 comprising a polynucleotide which comprises a sequence encoding a polypeptide that is at least 94%, preferably at least 95%, more preferably at least 96%, still more preferably at least 97%, yet more preferably at least 98%, and most preferably at least 99% identical to the sequence of SEQ ID NO: 1.

As used herein, it is in particular understood that the term “identical to the sequence of SEQ ID NO: X” is equivalent to the term “identical to the sequence of SEQ ID NO: X over the length of SEQ ID NO: X” or to the term “identical to the sequence of SEQ ID NO: X over the whole length of SEQ ID NO: X”, respectively. In this context, “X” is any integer selected from 1 to 2 so that “SEQ ID NO: X” represents any of the SEQ ID NOs mentioned herein.

Preferably, the infection with PCV2b, as described herein, is an infection with (i) a PCV2 comprising a polypeptide that is at least 94%, preferably at least 95%, more preferably at least 96%, still more preferably at least 97%, yet more preferably at least 98%, and most preferably at least 99% identical to the sequence of SEQ ID NO: 2 or (ii) a PCV2 comprising a polynucleotide which comprises a sequence encoding a polypeptide that is at least 94% %, preferably at least 95%, more preferably at least 96%, still more preferably at least 97%, yet more preferably at least 98%, and most preferably at least 99% identical to the sequence of SEQ ID NO: 2.

Particularly, in the context of the present invention, a PCV2a ORF2 protein or immunogenic composition comprising PCV2a ORF2 protein is preferred, wherein said PCV2a ORF2 protein is a recombinant PCV2a ORF2 protein, more preferably a recombinant baculovirus expressed PCV2a ORF2 protein.

In particular, for the purpose of the present invention, a PCV2a ORF2 protein or immunogenic composition comprising PCV2a ORF2 protein is preferred, wherein said PCV2a ORF2 protein comprises of consists of a sequence that is at least 94%, preferably at least 95%, more preferably at least 96%, still more preferably at least 97%, yet more preferably at least 98%, and most preferably at least 99% identical to the sequence of SEQ ID No: 1.

Preferably, in the context of the present invention,
the treatment or prevention of an infection with PCV2 of a different subtype is based on or comprises or consists of the induction of an immune response against said PCV2 of a different subtype or the concurrent induction of an immune response against said PCV2 of a different subtype and PCV2a,
the clinical signs, as mentioned herein, are selected from the group consisting of lymphoid depletion, lymphoid inflammation, lymphoid colonization, positive IHC for PCV2 antigen of lymphoid tissue, viremia, nasal shedding, pyrexia, reduced average daily weight gain, lung inflammation, positive IHC for PCV2 antigen of lung tissue, increased mortality, and/or
the disease, as mentioned herein, is PMWS.

The term “prevention” or “reduction” or “preventing” or “reducing”, respectively, as used herein, means, but is not limited to a process which includes the administration of a PCV2 antigen, namely of the PCV2a ORF2 protein according to the invention which is included in the composition of the invention, to an animal, wherein said PCV2 antigen, when administered to said animal elicits or is able to elicit an immune response in said animal against PCV2. Altogether, such treatment results in reduction of the clinical signs of a disease caused by PCV2 or of clinical signs associated with PCV2 infection, respectively. More specifically, the term “prevention” or “preventing”, as used herein, means generally a process of prophylaxis in which an animal is exposed to the immunogenic composition of the present invention prior to the induction or onset of the disease process caused by PCV2.

Herein, “reduction of clinical signs associated with PCV2 infection” means, but is not limited to, reducing the number of infected subjects in a group, reducing or eliminating the number of subjects exhibiting clinical signs of infection, or reducing the severity of any clinical signs that are present in the subjects, in comparison to wild-type infection. For example, it should refer to any reduction of pathogen load, pathogen shedding, reduction in pathogen transmission, or reduction of any clinical sign symptomatic of PCV2 infection. Preferably these clinical signs are reduced in subjects receiving the composition of the present invention by at least 10% in comparison to subjects not receiving the composition and may become infected. More preferably, clinical signs are reduced in subjects receiving the composition of the present invention by at least 20%, preferably by at least 30%, more preferably by at least 40%, and even more preferably by at least 50%.

The term “reduction of viremia” means, but is not limited to, the reduction of PCV2 virus entering the bloodstream of an animal, wherein the viremia level, i.e. the number of PCV2 RNA copies per mL of blood serum or the number of plaque forming colonies per deciliter of blood serum, is reduced in the blood serum of subjects receiving the composition of the present invention by at least 50% in comparison to subjects not receiving the composition and may become infected. More preferably, the viremia level is reduced in subjects receiving the composition of the present invention by at least 90%, preferably by at least 99.9%, more preferably by at least 99.99%, and even more preferably by at least 99.999%.

As used herein, the term “viremia” is particularly understood as a condition in which PCV2 particles reproduce and circulate in the bloodstream of an animal.

In the context of the present invention, “lymphoid colonization” is particularly understood as the presence of PCV2b antigen found in lymphoid tissue. More particular, said presence of PCV2b antigen found in lymphoidal tissue is based on the PCV2b colonization of lymphoid tissues. Thus, the reduction, prevention or treatment of lymphoid colonization, as described herein, in particular relates to the reduction of PCV2b antigen found in lymphoid tissue and to the reduction, prevention or treatment of the PCV2b colonization of lymphoid tissues, respectively.

The term “animal”, as used herein, in particular relates to a mammal, preferably to swine, more preferably to a pig, most preferably to a piglet.

The invention also provides a method for the treatment or prevention of an infection with PCV2 of a subtype other than subtype a, for the reduction, prevention or treatment of clinical signs caused by an infection with PCV2 of a subtype other than subtype 2a or for the treatment or prevention of a disease caused by an infection with PCV2 of a subtype other than subtype 2a, comprising administering PCV2a ORF2 protein or an immunogenic composition comprising PCV2a ORF2 protein to an animal.

Also, the invention provides the use of PCV2a ORF2 protein or of an immunogenic composition comprising PCV2a ORF2 protein for the preparation of a medicament for the reduction, prevention or treatment of clinical signs caused by an infection with PCV2 of a subtype other than subtype 2a or for the treatment or prevention of a disease caused by an infection with PCV2 of a subtype other than subtype 2a.

Also, in the context of the method of the invention or the use of the invention, preferably
the prevention, reduction or treatment of an infection with PCV2 of a subtype other than subtype 2a is based on or comprises or consists of the induction of an immune response against said PCV2 of a subtype other than subtype 2a or the concurrent induction of an immune response against said PCV2 of a subtype other than subtype a and PCV2a,
the clinical signs, as mentioned herein, are selected from the group consisting of lymphoid depletion, lymphoid inflammation, lymphoid colonization, positive IHC for PCV2 antigen of lymphoid tissue, viremia, nasal shedding, pyrexia, reduced average daily weight gain, lung inflammation positive IHC for PCV2 antigen of lung tissue, increased mortality, and/or
the disease, as mentioned herein, is PMWS.

According to a particular preferred aspect of the invention, the PCV2a ORF2 protein or the immunogenic composition is administered only once.

Preferably, in the context of the present invention, the PCV2a ORF2 protein or the immunogenic composition is administered to an animal, preferably to a swine, more preferably to a pig, in particular preferably to a pig

Most preferably, the immunogenic composition as described herein is an immunogenic composition, wherein the virucidal activity of the immunogenic composition has been reduced.

The practice of the present invention will employ, unless otherwise indicated, conventional techniques of molecular biology, microbiology, recombinant DNA technology, protein chemistry and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); Protein purification methods—a practical approach (E. L. V. Harris and S. Angal, eds., IRL Press at Oxford University Press); and Handbook of Experimental Immunology, Vols. I-IV (D. M. Weir and C. C. Blackwell eds., 1986, Blackwell Scientific Publications).

Before describing further aspects of the present invention in detail, it is to be understood that this invention is not limited to particular DNA, polypeptide sequences or process parameters as such may, of course, vary. It is also to be understood that the terminology used herein is for the purpose of describing particular embodiments of the invention only, and is not intended to be limiting. It must be noted that, as used in this specification and the appended claims, the singular forms “a”, “an” and “the” include plural referents unless the content clearly dictates otherwise. Thus, for example, reference to “an antigen” includes a mixture of two or more antigens; reference to “an excipient” includes mixtures of two or more excipients, and the like.

The present aspect of the invention solves the problems inherent in the prior art and provides a distinct advance in the state of the art. Generally, the present aspect of the invention includes a method of producing a PCV-2 antigenic composition to be used for the purposes of the present invention comprising the steps of i) obtaining a first liquid containing a PCV-2 antigen, and ii) removing at least a portion of the first liquid from the PCV-2 antigen. It is in particular understood that the term “antigenic composition”, as used herein, is equivalent to the term “immunogenic composition”.

For purposes of the present aspect of the invention, a “first liquid” refers to liquid, aqueous, or fluid media typically used in combination with cells, antigens, immunogenic compositions, vaccines, and the like. Preferably, the first liquid comprises media from an antigenic composition; more preferably, the first liquid comprises or preferably consists of cell culture media used for the production of recombinant proteins in cultivated host cells. The cultivated host cells can be bacteria, yeasts, insect cells, animal cells, and mammalian cells, with insect and mammalian cells being particularly preferred. Thus the first fluid may comprise or consist of media for the cultivation of bacteria, yeast, insect cells, animal cells, or mammalian cells. Preferably, the cell media is serum free cell media, and most preferably the culture media is EX-CELL® 420 serum free media, when insect cells are used. EX-CELL® 420 is a complete medium that is protein-free and contains L-glutamine, and was developed and optimized for the serum-free growth of Sf9 and Sf21 insect cell lines.

A “second liquid”, for purposes of the present aspect of the invention, refers to any liquid normally used in combination with cells, antigen, immunogenic compositions, vaccines, and the like, which is different from the first liquid. Preferably, the second liquid is an aqueous solution, even more preferably a pharmaceutically acceptable solution, and even more preferably a buffer, such as a saline or phosphate buffer and the like. Most preferably, the second fluid is characterized by not being virucidal to any live virus or any live bacteria (herein, unless explicitly stated or apparent from the context the term “virucidal” is inclusive of bactericidal activity), when the live virus or live bacteria is cultivated in or stored in such a fluid.

“Portion”, for purposes of the present aspect of the invention, refers to any amount which does not encompass the entire amount. For example, a portion of liquid would be anything less than 100% of the volume of the liquid, such as 90% of the liquid, 80% of the liquid, 70% of the liquid, and all amounts between more than 0% and less than 100%.

A “PCV-2 antigen” refers to any composition of matter that comprises at least one PCV2a antigen that can induce, stimulate or enhance the immune response against PCV-2 infection, when administered to an animal, preferably to a pig. Thus, the term “PCV-2” as mentioned hereinafter, in particular relates to PCV2a. Preferably, the PCV-2 antigen is the whole PCV-2 virus, preferably in an inactivated form, a live modified or attenuated PCV-2 virus, a chimeric virus that comprises at least an immunogenic amino acid sequence of PCV-2, or any other polypeptide or component that comprises at least an immunogenic amino acid sequence of PCV-2, preferably ORF2. The terms “immunogenic protein”, “immunogenic polypeptide” or “immunogenic amino acid sequence” as used herein refer to any amino acid sequence of PCV-2, which elicits an immune response in a host against PCV-2. Preferably, such immunogenic protein, immunogenic polypeptide or immunogenic amino acid of PCV-2 is any one of those disclosed or provided in the international patent application WO2006/072065 (the contents and teachings of which are hereby incorporated by reference), or is any other PCV-2 polypeptide known in the art. For instance, a representative sequence of PCV-2 ORF2 DNA comprises the nucleotide sequence Genbank Accession No. AF086834 (SEQ ID NO: 3) and SEQ ID NO: 4.

However, it is understood by those of skill in the art that this sequence could vary by as much as 1-10% in sequence homology and still retain the antigenic characteristics that render it useful in immunogenic compositions. The antigenic characteristics of an immunological composition can be, for example, estimated by the challenge experiment as provided by Example 4 of WO06/072065. Moreover, the antigenic characteristic of a modified antigen is still retained, when the modified antigen confers at least 70%, preferably 80%, more preferably 90% or more of the protective immunity as compared to the PCV-2 ORF2 protein, encoded by the polynucleotide sequence of SEQ ID NO:3 or SEQ ID NO:4 as provided in WO06/072065. Further preferred PCV-2 ORF2 antigens are as follow:
i) a polypeptide comprising the sequence of SEQ ID NO: 5, SEQ ID NO: 6, SEQ ID NO: 9, SEQ ID NO: 10 or SEQ ID NO: 11 of WO06/072065;
ii) any polypeptide that is at least 80% homologous and/or identical to the polypeptide of i),
iii) any immunogenic portion of the polypeptides of i) and/or ii)
iv) the immunogenic portion of iii), comprising at least 5, preferably 8, more preferably 10 contiguous amino acids of any of the sequences of SEQ ID NO: 5, SEQ ID NO: 6, SEQ ID NO: 9, SEQ ID NO: 10 or SEQ ID NO: 11 of WO06/072065,
v) a polypeptide that is encoded by a DNA comprising the sequence of SEQ ID NO: 3 or SEQ ID NO: 4 of WO06/072065.
vi) any polypeptide that is encoded by a polynucleotide that is at least 80% homologous and/or identical to the polynucleotide of v),
vii) any immunogenic portion of the polypeptides encoded by the polynucleotide of v) and/or vi),
viii) the immunogenic portion of vii), wherein the polynucleotide coding for the immunogenic portion comprises at least 30 contiguous nucleotides included in the sequences of SEQ ID NO: 3 or SEQ ID NO: 4 of WO06/072065.

Preferably any of the immunogenic portions described above having the antigenic characteristics of PCV-2 ORF2 antigen that is encoded by the sequence of SEQ ID NO: 3 or SEQ ID NO: 4 of WO06/072065.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions that are not identical differ by conservative amino acid substitutions. However, conservative substitutions are not included as a match when determining sequence identity.

“Live” virus or bacterium, for purposes of the present aspect of the invention, refers to a virus or bacterium that is capable of replicating in a host. A preferred live virus and a preferred live bacterium of the present aspect of the invention are the PRRS virus and the Mycoplasma hyopneumonia bacterium, respectively. However, the term live virus or live bacterium is not limited to PRRS virus and Mycoplasma hypneumoniae, respectively.

The portion of the first liquid can be removed from the PCV-2 antigen by an exchange of the portion of the first liquid against a second liquid, wherein the second liquid is different from the first liquid (see definition of second fluid). Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the portion of the first liquid is removed from the PCV-2 antigen by an exchange of the portion of the first liquid against a second liquid, and wherein the second liquid is different from the first liquid. Preferably the exchange of the portion of the first liquid with the second liquid comprises the steps of a) adding the second liquid to the first liquid which contains the PCV-2 antigen and b) concentrating the PCV-2 antigen by removing a portion of the first and second liquids from the PCV-2 antigen. Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen by an exchange of at least a portion of the first liquid against a second liquid comprising the steps a) adding the second liquid to the first liquid which contains the PCV-2 antigen and b) concentrating the PCV-2 antigen by removing a portion of the first and second liquids from the PCV-2 antigen.

The portion of the first liquid can be removed from the PCV-2 antigen by a filtration step utilizing a filter. However, any other method known to a person skilled in the art can be used to remove the portion of any fluids, including the first and, whenever applicable, a portion of the second fluid from the PCV-2 antigen. Such method, for instance, includes but is not limited to centrifugation and/or chromatography. However, filtration is most preferred. A preferred filtration method to remove the portion of the first fluid, or any other fluid, whenever applicable, comprises ultra- and/or dia-filtration. Ultra- and dia-filtration are standard methods known to a person skilled in the art, described for example in detail in Protein Purification Methods—A Practical Approach—editors: E. L. V. Harris and S. Angel, Oxford University Press 1995 (the contents and teachings of which are hereby incorporated by reference). In particular, in Chapter 3 of that textbook, several methods and types of equipment are described, all of which can be used by an ordinary person skilled in the art in an exemplary manner for the purpose of the present aspect of the invention. Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the portion of the first liquid is removed from the PCV-2 antigen by filtration, preferably by dia- or ultra-filtration. Preferably, the portion of the first liquid is removed from the PCV-2 antigen by an exchange of at least a portion of the first liquid against a second liquid comprising the steps a) adding the second liquid to the first liquid which contains the PCV-2 antigen and b) concentrating the PCV-2 antigen by removing a portion of the first and second liquids from the PCV-2 antigen.

As defined above, a preferred second liquid to be used in any of the methods described is a buffer, preferably a physiologically acceptable buffer with saline being particularly preferred. Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, by an exchange against a buffer, preferably a physiologically acceptable buffer such as saline or phosphate buffer or the like. Preferably the portion of the first liquid is removed from the PCV-2 antigen by filtration, preferably by dia- and/or ultra-filtration. More preferably, the portion the exchange of at least a portion of the first liquid against the buffer, preferably the physiologically acceptable buffer, such as saline or phosphate buffer or the like, comprising the steps a) adding the buffer, preferably the physiologically acceptable buffer, such as saline or phosphate buffer or the like, to the first liquid which contains the PCV-2 antigen and b) concentrating the PCV-2 antigen by removing a portion of the first and the fluid which is a buffer, preferably a physiologically acceptable buffer, such as saline or phosphate buffer or the like, from the PCV-2 antigen, preferably by filtration, even more preferably by dia- and/or ultra-filtration.

The concentrating step and the liquid addition step of the method as described herein can be performed substantially simultaneously or alternatively, the concentrating step and the liquid addition step are performed sequentially. Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen by an exchange of a portion of the first liquid against a second liquid comprising the steps of a) adding the second liquid to the first liquid which contains the PCV-2 antigen and b) concentrating the PCV-2 antigen by removing a portion of the first and second liquids from the PCV-2 antigen, wherein the liquid addition step is performed substantially simultaneously or sequentially. Preferably the portion of the first liquid and in the case of the addition of the second liquid, the mixture of the first and the second fluid is removed from the PCV-2 antigen by filtration, preferably by dia- and/or ultra-filtration.

When the concentrating step and liquid addition step are performed sequentially, the order of the steps does not matter. For example, in a further aspect, the liquid addition step occurs prior to the concentrating step and in an alternative aspect, the concentrating step occurs prior to the liquid addition step. The liquid addition step and the concentrating step, regardless of the order in which they are performed, can be performed multiple times. For example, each of these respective steps can be performed at least two, at least three, at least four, at least five, at least 10, up to as many times as desired. In one aspect, the concentrating step and the liquid addition step are each performed at least two times. In another aspect, the concentrating step and the liquid addition step are each performed at least three times. Thus, according to a further aspect of the present aspect of the invention, a method of producing a PCV-2 antigenic composition is provided wherein the method generally comprises the steps of i) obtaining a first liquid containing a PCV-2 antigen, ii) removing at least a portion of the first liquid from the PCV-2 antigen by an exchange of the portion of the first liquid against a second liquid, wherein the exchange is performed multiple times. Preferably the exchange of the portion of the first fluid against a portion of the second fluid comprises the steps of a) adding the second liquid to the first liquid which contains the PCV-2 antigen and b) concentrating the PCV-2 antigen by removing a portion of the first and second liquids from the PCV-2 antigen, wherein the liquid addition step and concentration step are performed multiple times, for instance, two times, three times, 5 times, 10 times, etc. Preferably, the liquid addition step and concentration step are performed two times, most preferably three times. As described above, filtration is the preferred method to remove a portion of the first liquid, or in case of multiple removing steps as described above, to remove a portion of the mixture of the first and the second fluid, from the PCV-2 antigen.

The filter can be any conventional filter in the art. Preferably, the filter includes a semi-permeable membrane. In a further preferred form, the semi-permeable membrane has an average pore size that is smaller than the PCV-2 antigen to thereby prevent passage of at least 90% of the PCV-2 antigen through the semi-permeable membrane pores and withhold the PCV-2 antigen by the filter. In a further aspect, the filter has an average pore size which prevents passage of at least 90% of proteins of 50 kDa to 500 kDa in size, more preferably, the filter has an average pore size which prevents passage of at least 90% of proteins of 75 kDa to 400 kDa in size, and most preferably, the filter has an average pore size which prevents passage of at least 90% of proteins of 100 kDa to 300 kDa in size. This pore size is preferred, when the PCV-2 antigen is produced as whole virus or as virus like particles. In a still further aspect, the semi-permeable membrane includes a material selected from the group consisting of polysulfone, polyethersulfone, and regenerated cellulose. However, any other material that allows removing of a portion of the first fluid, and in case of a multiple process step, removing of a mixture of the first and the second fluid from the PCV-2 antigen can be used. The filter can be selected from the group consisting of a hollow fiber membrane ultra filtration cartridge, flat sheets, or a cassette, with a hollow fiber membrane ultra filtration cartridge being particularly preferred. Thus, according to a further aspect of the present aspect of the invention, a method of producing a PCV-2 antigenic composition is provided as described above. The method generally comprises the steps of i) obtaining a first liquid containing a PCV-2 antigen, ii) removing at least a portion of the first liquid from the PCV-2 antigen by a filtration step, wherein the filter preferably is or comprises a semi-permeable membrane. Preferably, the semi-permeable membrane has an average pore size that is smaller than the PCV-2 antigen and prevents passage of at least 90% of the PCV-2 antigen through the semi-permeable membrane pores. Preferably the average pore size of the semi-permeable membrane prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. As described above, the removing step in general includes the exchange of the portion of the first fluid against a portion of the second fluid comprising the steps of a) adding the second liquid to the first liquid which contains the PCV-2 antigen and b) concentrating the PCV-2 antigen by removing a portion of the first and second liquids from the PCV-2 antigen, wherein the liquid addition step and concentration step are performed multiple times, for instance, two times, three times, 5 times, 10 times, etc. Preferably, the liquid addition step and the concentration step are performed two times, most preferably three times.

The concentration step of the method provided herein is performed such that the PCV-2 antigen is concentrated from 3× to 50× in comparison to the volume of the first liquid. More preferably, the concentrating step is done such that the PCV-2 antigen is concentrated 4× to 20× in comparison to the volume of the first liquid. Most preferably, concentration step is done such that the PCV-2 antigen is concentrated from 7× to 10× in comparison to the volume of the first liquid. Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the portion of the first liquid is removed from the PCV-2 antigen, and wherein the PCV-2 antigen is concentrated from 3× to 50×, preferably from 4× to 20×, and even more preferably from 7× to 10× in comparison to the volume of the first liquid. Preferably, the portion of the first fluid is removed from the PCV-2 antigen by an exchange of the portion of the first liquid against a second liquid comprising the steps of a) adding the second liquid to the first liquid which contains the PCV-2 antigen and b) concentrating the PCV-2 antigen from 3× to 50×, preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, even more preferably three times. In such case, not only the first liquid is removed, but also a mixture of the first and second liquid. Preferably each liquid addition step is performed substantially simultaneously or sequentially. When the concentrating step and liquid addition step are performed sequentially, the order of the steps does not matter. Moreover, the concentration step is preferably done by filtration—preferably dia- and/or ultra-filtration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores. Preferably the average pore size of the semi-permeable membrane is prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

In a further aspect, the virucidal activity of the PCV-2 antigenic composition produced by the methods herein is reduced by at least 10% as compared to the liquid that has not undergone the method. More preferably, the virucidal activity of the PCV-2 antigenic composition is reduced by at least 50% as compared to the first liquid that has not undergone the method. Still more preferably, the virucidal activity of the PCV-2 antigenic composition is reduced by at least 70% as compared to the first liquid that has not undergone the method.

For the purpose of the current aspect of the invention the term “virucidal activity” means, that a fluid, solution or composition inactivates or kills a live virus or live bacteria to a certain extent, when the fluid, solution or composition is mixed with such live virus or live bacteria. Thus, a reduction of the virucidal activity of a fluid, solution or composition by at least 10% means, that the survival rate of a live virus or live bacteria is 90% higher in a fluid, solution or composition that has undergone any of the methods described herein, as compared to a fluid, solution or composition, that has not undergone any of the method described herein. According to the present aspect of the invention, the PRRS virus, preferably PRRS virus having the ATCC accession number VR 2332, is the reference virus for the determination of virucidal activity. To determine the virucidal activity with regard to a bacterium, it is proposed to use the Mycoplasma hyopneumonia bacterium, preferably the J-strain of Mycoplasma hyopneumonia.

Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the virucidal activity—preferably in respect to PRRS virus—of the PCV-2 antigenic composition obtained after step ii) is reduced by at least 10%, preferably at least 50%, more preferably at least 70%, even more preferably at least 90% as compared to that of the first liquid. Preferably, the portion of the first liquid having virucidal activity is removed from the PCV-2 antigen by an exchange of a portion of the first liquid against a second liquid. The exchange is preferably done in such a manner that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, and even more preferably three times. In such case, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultra-filtration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

In a further aspect, the method further comprises the step of harvesting the PCV-2 antigen obtained after at least a portion of the first liquid is removed from the PCV-2 antigen.

As used herein, “harvesting” or “harvest” refers to the collecting or recovering of the PCV-2 antigen. Any conventional method known in the art can be used to recover the PCV-2 antigen either when an antigen is being produced for use with the methods and compositions of the present aspect of the invention, or when the PCV-2 antigen is undergoing the methods described herein. In a particularly preferred manner of harvesting, the portion of the first liquid is removed from the PCV-2 antigen via a filtration step and the PCV-2 antigen is recovered or harvested from the filter retard. In a more preferred form, the PCV-2 antigen is harvested or collected, or recovered from the retard of a semi-permeable membrane having the pore size described herein. Thus, according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the PCV-2 antigen obtained after the step ii) is harvested. Preferably, the portion of the first liquid is removed from the PCV-2 antigen by an exchange of a portion of the first liquid against a second liquid. The exchange is preferably done such that it comprises the steps of a) adding a second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultra-filtration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

The PCV-2 antigen remaining after undergoing the methods provided herein, preferably after being harvested from the filter retard, is admixed with a further component selected from the group consisting of pharmaceutically acceptable carriers, adjuvants, diluents, excipients, and combinations thereof. Preferably, the further component is an adjuvant, even more preferably wherein the adjuvant is a polymer of acrylic or methacrylic acid, and still more preferably wherein the adjuvant is Carbomer (the generic name for synthetic high molecular weight polymers of acrylic acid).

As used herein, “a pharmaceutical-acceptable carrier” and a “veterinary acceptable carrier” includes any and all solvents, dispersion media, coatings, stabilizing agents, diluents, preservatives, antibacterial and antifungal agents, isotonic agents, adsorption delaying agents, and the like.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). JohnWiley and Sons, NY, pp 51-94 (1995) and Todd et al., Vaccine 15:564-570 (1997). For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book. Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others. Among the copolymers of maleic anhydride and alkenyl derivative, the copolymers EMA (Monsanto), which are copolymers of maleic anhydride and ethylene, are included. The dissolution of these polymers in water leads to an acid solution that will be neutralized, preferably to physiological pH, in order to give the adjuvant solution into which the immunogenic, immunological or vaccine composition itself will be incorporated.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name CARBOPOL®; (BF Goodrich, Ohio, USA) are particularly appropriate. They are polymers of acrylic acid cross-linked with polyalkenyl ethers or divinyl glycol or cross-linked with allyl sucrose or with allyl pentaerythritol. Among them, there may be mentioned CARBOPOL® 974P, 934P and 971P. Most preferred is the use of CARBOPOL® 971P.

Preferably, the adjuvant is added in an amount of about 100 μg to about 10 mg per dose. Even more preferably the adjuvant is added in an amount of about 100 μg to about 10 mg per dose. Still more preferably the adjuvant is added in an amount of about 500 μg to about 5 mg per dose. Still more preferably the adjuvant is added in an amount of about 750 μg to about 2.5 mg per dose. Most preferably the adjuvant is added in an amount of about 1 mg per dose.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

A “preservative” as used herein refers to an anti-microbiological active agent, such as for example Gentamycin, Merthiolate, and the like. In particular adding of a preservative is most preferred for the preparation of a multi-dose composition. Those anti-microbiological active agents are added in concentrations effective to prevent the composition of interest for any microbiological contamination or for inhibition of any microbiological growth within the composition of interest.

Thus, according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, further comprising the step of admixing the PCV-2 antigen remaining after step ii) with a further component selected from the group consisting of pharmaceutically acceptable carriers, adjuvants, diluents, excipients, and combinations thereof. Preferably wherein the further component is an adjuvant, even more preferably wherein the adjuvant is a polymer of acrylic or methacrylic acid, and still more preferably wherein the adjuvant is Carbomer. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and the concentration step are performed multiple times, preferably two times, and even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

The PCV-2 antigen used in the methods described above can be any PCV-2 antigen as defined herein. Preferably the PCV-2 antigen comprises the ORF-2 protein of PCV-2, more preferably recombinant ORF-2 protein of PCV-2, and still more preferably virus like particles of ORF-2 protein, and even more preferably the antigen included in INGELVAC CIRCOFLEX®. Thus, according to a further aspect of the present application, the present application provides a method of producing a PCV-2 antigenic composition comprising the steps of i) obtaining a first liquid containing a PCV-2 antigen, ii) removing at least a portion of the first liquid from the PCV-2 antigen, wherein the PCV-2 antigen comprises the ORF-2 protein of PCV-2, more preferably recombinant ORF-2 protein of PCV-2, and still more preferably virus like particles of ORF-2 protein. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen.

Preferably, the liquid addition step and concentration step are performed multiple times, preferably two times, and even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

The first liquid containing the PCV-2 antigen used can be obtained by any method known in the art. Preferably, the first liquid containing the PCV-2 antigen as well as PCV-2 antigen can be obtained by any of the methods described in the international patent application WO2006/072065 (the contents and teachings of which are hereby incorporated by reference). In particular, the PCV-2 antigen, when expressed recombinantly in vitro in host cells, can be obtained via a viral vector, preferably a recombinant baculovirus viral vector, containing and expressing the PCV-2 antigen, preferably, PCV-2 ORF-2.

Vectors and methods for making and/or using vectors (or recombinants) for expression of the PCV-2 antigen, preferably the PCV-2 ORF2 antigen can be by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 382,425, PCT publications WO 94/16716, WO 96/39491, WO 95/30018, Paoletti, “Applications of pox virus vectors to vaccination: An update,” PNAS USA 93: 11349-11353, October 1996, Moss, “Genetically engineered poxviruses for recombinant gene expression, vaccination, and safety,” PNAS USA 93: 11341-11348, October 1996, Smith et al., U.S. Pat. No. 4,745,051, (recombinant baculovirus), Richardson, C. D. (Editor), Methods in Molecular Biology 39, “Baculovirus Expression Protocols” (1995 Humana Press Inc.), Smith et al., “Production of Human Beta Interferon in Insect Cells Infected with a Baculovirus Expression Vector”, Molecular and Cellular Biology, December, 1983, Vol. 3, No. 12, p. 2156-2165; Pennock et al., “Strong and Regulated Expression of Escherichia coli B-Galactosidase in Infect Cells with a Baculovirus vector, “Molecular and Cellular Biology March 1984, Vol. 4, No. 3, p. 399-406; EPAO 370 573, U.S. application No. 920,197, filed Oct. 16, 1986, EP Patent publication No. 265785, U.S. Pat. No. 4,769,331 (recombinant herpesvirus), Roizman, “The function of herpes simplex virus genes: A primer for genetic engineering of novel vectors,” PNAS USA 93:11307-11312, October 1996, Andreansky et al., “The application of genetically engineered herpes simplex viruses to the treatment of experimental brain tumors,” PNAS USA 93: 11313-11318, October 1996, Robertson et al. “Epstein-Barr virus vectors for gene delivery to B lymphocytes”, PNAS USA 93: 11334-11340, October 1996, Frolov et al., “Alphavirus-based expression vectors: Strategies and applications,” PNAS USA 93: 11371-11377, October 1996, Kitson et al., J. Virol. 65, 3068-3075, 1991; U.S. Pat. Nos. 5,591,439, 5,552,143, WO 98/00166, allowed U.S. application Ser. Nos. 08/675,556, and 08/675,566 both filed Jul. 3, 1996 (recombinant adenovirus), Grunhaus et al., 1992, “Adenovirus as cloning vectors,” Seminars in Virology (Vol. 3) p. 237-52, 1993, B allay et al. EMBO Journal, vol. 4, p. 3861-65, Graham, Tibtech 8, 85-87, April, 1990, Prevec et al., J. Gen Virol. 70, 42434, PCT WO 91/11525, Felgner et al. (1994), J. Biol. Chem. 269, 2550-2561, Science, 259: 1745-49, 1993 and McClements et al, “Immunization with DNA vaccines encoding glycoprotein D or glycoprotein B, alone or in combination, induces protective immunity in animal models of herpes simplex virus-2 disease”, PNAS USA 93: 11414-11420, October 1996, and U.S. Pat. Nos. 5,591,639, 5,589,466, and 5,580,859, as well as WO 90/11092, WO93/19183, WO94/21797, WO95/11307, WO95/20660, Tang et al., Nature and Furth et al. Analytical Biochemistry, relating to DNA expression vectors, inter alia. See also WO 98/33510; Ju et al., Diabetologia, 41: 736-739, 1998 (lentiviral expression system); Sanford et al., U.S. Pat. No. 4,945,050; Fischbach et al. (Intracel), WO 90/01543; Robinson et al., seminars in Immunology vol. 9, pp. 271-283 (1997), (DNA vector systems); Szoka et al., U.S. Pat. No. (method of inserting DNA into living cells); McCormick et al., U.S. Pat. No. 5,677,178 (use of cytopathic viruses); and U.S. Pat. No. 5,928,913 (vectors for gene delivery), as well as other documents cited herein. The expression of PCV-2 ORF2 antigen in insect cells is described, for instance, in WO 06/072065. The purified PCV-2 ORF2 antigen according to the aspect of the invention can be obtained by several methods known in the art. Preferred methods are those described herein. The PCV-2 ORF2 antigen can be produced recombinantly in vitro by the method comprising the steps i) permitting infection of susceptible cells in culture with a recombinant viral vector containing PCV-2 ORF2 coding sequence, wherein the PCV-2 ORF2 protein is expressed by the recombinant viral vector, and ii) thereafter recovering the PCV-2 ORF2 antigen from cell culture. The PCV-2 ORF2 antigen is recovered by harvesting the whole (i.e. intact) SF+ cells expressing the PCV-2 ORF2 antigen.

Thus, according to a further aspect of the present application, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the PCV-2 antigen is obtained via a viral vector, preferably a recombinant baculovirus viral vector, containing and expressing the PCV-2 antigen, preferably, PCV-2 ORF-2, and wherein the PCV-2 antigen comprises the ORF-2 protein of PCV-2, more preferably recombinant ORF-2 protein of PCV-2, and still more preferably virus like particles of ORF-2 protein. When a viral vector, in particular a recombinant baculovirus containing and expressing the PCV-2 antigen is used to produce/obtain the PCV-2 antigen, the method described above further comprises the step of inactivating the viral vector, preferably the recombinant baculovirus viral vector with a DNA inactivating agent, preferably in the presence of about 1 to about 20 mM of binary ethylenimine. Preferably, the inactivating step is performed after at least a portion of the first liquid is removed from the PCV-2 antigen, more preferably after the PCV-2 antigen is harvested. Even more preferably, the inactivating step is performed after the portion of the first liquid is removed from the PCV-2 antigen by an exchange of a portion of the first liquid against a second liquid. When the exchange of a portion of the first liquid against a second liquid is done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen, the inactivating step is done after the concentration step. When the liquid addition step and concentration step are performed multiple times, preferably two times, even more preferably three times, the inactivation step is performed after the last liquid addition step and concentration step. When the concentration step is done by filtration—preferably by dia- and/or ultra-filtration, utilizing a filter, preferably containing a semi-permeable membrane, the inactivation step is performed after the filtration step described above, preferably utilizing a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

“DNA inactivating agent”, for purposes of the present aspect of the invention, refers to any chemical agent which deactivates the DNA, preferably, DNA of a pathogen, such that the pathogen cannot cause active infection or be infective or replicate, but is still capable of inducing an immune response in a subject. Preferably, the DNA inactivating agent is formalin.

Thus, according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the PCV-2 antigen is obtained via a viral vector, preferably a recombinant baculovirus viral vector, containing and expressing the PCV-2 antigen, preferably, PCV-2 ORF-2, wherein the method further comprises the step of inactivating the viral vector, preferably the recombinant baculovirus viral vector with a DNA inactivating agent, preferably in the presence of about 1 to about 20 mM of binary ethylenimine, and wherein the PCV-2 antigen comprises the ORF-2 protein of PCV-2, more preferably recombinant ORF-2 protein of PCV-2, and still more preferably virus like particles of ORF-2 protein. Preferably, the inactivating step is performed after at least a portion of the first liquid is removed from the PCV-2 antigen, more preferably after the PCV-2 antigen is harvested. Even more preferably, the inactivating step is performed after the portion of the first liquid is removed from the PCV-2 antigen by an exchange of a portion of the first liquid against a second liquid. When the exchange of a portion of the first liquid against a second liquid is done such that it comprises the steps of a) adding the second liquid to the first liquid which contains the PCV-2 antigen and b) concentrating the PCV-2 antigen, preferably from 3× to 50×, even more preferably from 4× to 20×, even more preferably from 7× to 10× in comparison to the volume of the first liquid by removing a portion of the first and second liquids from the PCV-2 antigen, the inactivating step is done after the concentration step. When the liquid addition step and concentration step are performed multiple times, preferably two times, even more preferably three times, such inactivation step is performed after the last liquid addition step and concentration step. When the concentration step is done by filtration—preferably by dia- and/or ultra-filtration, utilizing a filter, preferably containing a semi-permeable membrane, the inactivation step is performed after the filtration step described above, preferably utilizing a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

In the case that a DNA inactivating agent is used in the method according to the aspect of the invention the method further comprises the step of adding an amount of an agent that neutralizes the DNA inactivating agent, the amount being equivalent to the amount of the DNA inactivating agent wherein the agent that neutralizes the DNA inactivating agent comprises a sodium thiosulfate solution concentrated to a final concentration of about 1 to about 20 mM and wherein the DNA inactivating agent is BEI. Preferably, the inactivating step is performed after at least a portion of the first liquid is removed from the PCV-2 antigen.

“Agent that neutralizes the inactivating agent” or “neutralizing agent”, as used herein, refers to any agent capable of neutralizing the inactivating agents listed above such that the inactivating agent is no longer capable of inactivating DNA. The agent that neutralizes the inactivating agent is preferably sodium thiosulfate.

Thus, according to a further aspect, the present aspect of the invention provides a method of producing a PCV-2 antigenic composition comprising the steps of i) obtaining a PCV-2 antigen in a first liquid wherein the PCV-2 antigen is obtained via a viral vector, preferably a recombinant baculovirus viral vector, containing and expressing the PCV-2 antigen, preferably, PCV-2 ORF-2, and wherein the PCV-2 antigen comprises the ORF-2 protein of PCV-2, more preferably recombinant ORF-2 protein of PCV-2, and still more preferably virus like particles of ORF-2 protein; ii) removing at least a portion of the first liquid from the PCV-2 antigen; iii) inactivating the recombinant baculovirus viral vector with a DNA inactivating agent, preferably in the presence of about 1 to about 20 mM of binary ethylenimine; iv) adding an amount of a neutralizing agent that neutralizes the inactivating agent, the amount of neutralizing agent being equivalent to the amount of the inactivating agent, wherein the neutralizing agent preferably comprises a sodium thiosulfate solution preferably concentrated to a final concentration of about 1 to about 20 mM and wherein the inactivating agent preferably comprises BEI. Preferably, the inactivating and neutralization step is performed after at least a portion of the first liquid is removed from the PCV-2 antigen, more preferably after the PCV-2 antigen is harvested. Even more preferably, the inactivating and neutralization step is performed after the portion of the first liquid is removed from the PCV-2 antigen by an exchange of a portion of the first liquid against a second liquid. When the exchange of a portion of the first liquid against a second liquid is done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen, the inactivating and neutralization step is done after the concentration step. When the liquid addition step and concentration step are performed multiple times, preferably two times, even more preferably three times, the inactivation and neutralization step is performed after the last liquid addition step and concentration step. When the concentration step is done by filtration—preferably by dia- and/or ultrafiltration, utilizing a filter, preferably containing a semi-permeable membrane, the inactivation and neutralization step is performed after the filtration step described above, preferably utilizing a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

In a further aspect of the present aspect of the invention, the method described above further comprises the steps admixing the PCV-2 antigen obtained after the inactivating and neutralizing steps with a further component selected from the group consisting of pharmaceutically acceptable carriers, adjuvants, diluents, excipients, and combinations thereof. Thus, according to a further aspect, the present aspect of the invention provides a method of producing a PCV-2 antigenic composition comprising the steps of i) obtaining a PCV-2 antigen in a first liquid, wherein the PCV-2 antigen is obtained via a viral vector, preferably a recombinant baculovirus viral vector, containing and expressing the PCV-2 antigen, preferably, PCV-2 ORF-2, and wherein the PCV-2 antigen comprises the ORF-2 protein of PCV-2, more preferably recombinant ORF-2 protein of PCV-2, and still more preferably virus like particles of ORF-2 protein; ii) removing at least a portion of the first liquid from the PCV-2 antigen; iii) inactivating the recombinant baculovirus viral vector with a DNA inactivating agent, preferably in the presence of about 1 to about 20 mM of binary ethylenimine; iv) adding an amount of a neutralizing agent that neutralizes the inactivating agent, the amount of neutralizing agent preferably being equivalent to the amount of the inactivating agent, wherein the neutralizing agent preferably comprises a sodium thiosulfate solution preferably concentrated to a final concentration of about 1 to about 20 mM and wherein the inactivating agent preferably comprises BEI; and v) admixing the PCV-2 antigen obtained in step iv) with a further component selected from the group consisting of pharmaceutically acceptable carriers, adjuvants, diluents, excipients, and combinations thereof. Preferably, the PCV-2 antigen comprises the ORF-2 protein of PCV-2, more preferably recombinant ORF-2 protein of PCV-2, and still more preferably virus like particles of ORF-2 protein. Preferably, in step ii), the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, even more preferably three times. In such case, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

According to a further aspect, any of the method described above to obtain a PCV-2 antigen with reduced virucidal activity can include further purification steps to obtain a purified PCV-2 antigen. It was surprisingly found that an antigenic or immunogenic composition comprising a purified PCV-2 antigen, preferably in combination with an adjuvant, not only shows a reduced virucidal activity as described herein, but also shows an increased immunogenicity as compared to an immunogenic composition, which does not comprise a purified PCV-2 antigen, means which comprises a non-purified or crude PCV-2 antigen.

The term “purified PCV-2 antigen” means, that the PCV-2 antigen is purified in a preparation to an extent of more than 50% (w/w), preferably of more than 60% (w/w), preferably of more than 70% (w/w), preferably of more than 80% (w/w), preferably of more than 85% (w/w), more preferably of more than 90% (w/w), even more preferable of more than 95% (w/w) with reference to the total amount of protein included in the immunogenic composition. In other words, if a preparation comprises a PCV-2 antigen with purity grade of 80% (w/w), such preparation comprise not more than 20% (w/w) of non PCV-2 proteins with reference to the total amount of protein included in the immunogenic composition. Preferably, the grade of purity is measured in the preparation, i.e. in the immunogenic composition before admixing with adjuvant or any other excipients or inactivating agent. However, if the adjuvant used in the final immunogenic composition is a non-protein based adjuvant, the addition of the adjuvant does not have any effect of the purity value. The purity grade of the PCV-2 antigen can be estimated by standard methods known to a person skilled in the art, for instance by Imperial Protein Stain (Pierce) after SDS-PAGE separation, gas chromatography, HPLC analyses, etc. The preferred method according to this aspect of the invention to estimate the purity or purity grade of a PCV-2 antigen in a preparation i.e. an immunogenic composition is the Imperial Protein Stain (Pierce) staining, which is done as follows: The preparation comprising the PCV-2 antigen are separated via NuPAGE 10% Bis-Tris gels (Invitrogen) using the NuPAGE MOPS buffer system (Invitrogen). Gels were run under denaturing (all buffers have SDS in them) and reducing conditions (the loading buffer has 2-mercaptoethanol). After loading the gels with samples, the gels were run for 55 min at 200 Volts constant. Once the run was completed, the gels were stained using Imperial Protein Stain (Pierce) and destained according the manufacturer's instructions.

In contrast, the term “non-purified” or “crude” PCV-2 antigen refers to a crude preparation comprising PCV-2 antigen. PCV-2 antigen is normally produced in vitro in cell culture. Thus, a crude PCV-2 antigen refers to a mixture of PCV-2 antigen and the cell culture or cell culture material used for the production of the PCV-2 antigen. Moreover, a non-purified PCV-2 antigen also means a partial purified PCV-2 antigen, preferably having a purity grade of less than 50% (w/w), more preferred of less than 40% (w/w), even more preferred of less than 30% (w/w), even more preferred of less than 20% (w/w) with reference to the total amount of protein included in the immunogenic composition.

In addition, the terms “increased immunogenicity or improved immunogenicity” as used herein, mean that the immune response caused by an immunogenic composition comprising an antigen of interest is increased as compared to a reference immunogenic composition comprising a different antigen or different purity grade of the antigen, whether this immune response is a cellular mediated and/or antibody mediated immune response. According to a preferred embodiment, the term increased immunogenicity or improved immunogenicity means, that the antibody mediated immune response elicited by an immunogenic composition comprising the antigen of interest is increased as compared to a reference immunogenic composition comprising a different antigen or a different purity grade of the antigen. In this regard antibody mediated immune response means, that the production of antibodies, which are specific to the antigen of interest is increased as compared to the antibody production elicited by a reference immunogenic composition comprising a different antigen or a different purity grade of the antigen.

The term “increased” means, that the cellular and/or antibody mediated immune response is increased by at least 10%, preferably by at least 20%, more preferably by at least 30%, even more preferably by at least 40%, even more preferably by at least 50%, even more preferably by at least 75%, most preferably by at least 100% as compared to the cellular and/or antibody mediated immune response elicited by a reference immunogenic composition comprising a different antigen or a different purity grade of the antigen.

It is in the general knowledge of a person skilled in the art how to measure the cellular and/or antibody mediated immune response. In particular, it is clear to such person skilled in the art either to compare the cellular mediated immune response of the immunogenic composition of interest with cellular mediated immune response of the reference, or the antibody mediated immune response of the immunogenic composition of interest with that of the reference composition, but neither the cellular mediated immune response of a immunogenic composition of interest with the antibody mediated immune response of the reference or vice versa. Moreover, the cellular mediated immune response can be measured, for instance, by measuring the activation of cytotoxic T-cells by an immunogenic composition/antigen of interest. The antibody mediated immune response can be measured, for instance, by measuring the amount of antigen specific antibodies, generated in cause of the administration of the immunogenic composition comprising such antigen to an animal. The cellular and/or antibody mediated immune response can be measured, for instance, by using a mouse model. According to the current aspect of the invention, the mouse model is used as the reference method.

The term “immunogenic composition” means, but is not limited to, a composition of matter that comprises at least one antigen which elicits a cellular and/or antibody-mediated immune response in a host against the antigen of interest. Usually, an “immune response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Preferably, the host will display either a therapeutic or protective immune response such that resistance to new infection will be enhanced and/or the clinical severity of the disease reduced. In such a case the immunogenic composition is a “vaccine”. Such protection will be demonstrated by either a reduction or lack of symptoms normally displayed by an infected host, a quicker recovery time and/or a lowered viral titer in the infected host.

Further purification of the PCV-2 antigen can be achieved with chromatography procedures, preferably a two-step chromatography procedure. If the PCV-2 antigen is assembled to virus like particles (VLP), one step, preferably the first step, is preferably a size exclusion (gel filtration) chromatography, which can be done, for instance, by using a Sephacryl 5300 matrix. In lab scale use of HiPrep 26/60 Sephacryl S300HR columns are most preferred. However, any other size exclusion chromatography matrices known to a person skilled in the art can be used, which allow the separation of the PCV-2 ORF2 VLPs from the culture filtrate or supernatant. Suitable matrices are described, for instance, in E. L. V. Harris and S. Angel (eds.), Protein purification methods—a practical approach, IRL Press Oxford 1995). The gel filtration chromatography can be conducted, for instance, by loading the column with the crude preparation comprising the PCV-2 antigen with a flow-rate of 1.0 ml/min and eluting the column with 1.5 column volume of a buffer comprising 20 mM Tris, pH 6.5, 5 mM DTT. However, the PCV-2 ORF2 antigen can also be purified by using affinity chromatography, for instance, via selective binding to an immobilized PCV-2 ORF2 specific antibody, or any other method known to a person skilled in the art.

Thus according to a preferred embodiment the present aspect of the invention provides a method of producing a PCV-2 antigenic composition comprising the steps of i) obtaining a first liquid containing a PCV-2 antigen, ii) removing at least a portion of the first liquid from the PCV-2 antigen and iii) purifying the harvest of step ii) comprising the PCV-2 antigen, preferably the PCV-2 ORF2 antigen by chromatographic procedure. Preferably size exclusion chromatography is performed as described herein, preferably as described in Example 3. Preferably, the size exclusion results in an immunogenic composition having purity grade of more than 80% (w/w), preferably more than 90% (w/w) with reference to the total amount of protein included in the immunogenic composition prior to the mixture with the adjuvant. The purity grade can be estimated by Imperial Protein Stain (Pierce) staining after SDS PAGE via NuPAGE 10% Bis-Tris gels (Invitrogen) using the NuPAGE MOPS buffer system (Invitrogen).

Thus according to a preferred embodiment the present aspect of the invention provides a method of producing a PCV-2 antigenic composition comprising the steps of i) obtaining a first liquid containing a PCV-2 antigen, ii) removing at least a portion of the first liquid from the PCV-2 antigen and iii) purifying the harvest of step ii) comprising the PCV-2 antigen by size exclusion chromatography (gel filtration).

In order to obtain a higher purity grade a second chromatography step can be done, which however is different from the first one. For instance if the first purification step/chromatography step is size exclusion (gel filtration) the second should different from that e.g. an affinity chromatography, ion exchange chromatography, etc. Preferably, if the first step to purify PCV-2 antigen, preferably to purify PCV-2 ORF2 antigen is a size exclusion (gel filtration) chromatography, the second step can be ion-exchange chromatography, preferably anion-exchange chromatography (AIEX). A preferred anion-exchange chromatography matrix for the purification of PCV-2 antigen, preferably the PCV-2 ORF2 antigen is Q Sepharose. In a small scale of about 50 ml, use of 5 ml HiTrap Q Sepharose HP columns are most preferred. The anion exchange chromatography can be conducted, for instance, as described in Example 3. Briefly, about 50 ml of the void volume fraction pool from the size exclusion chromatography step can be loaded onto the AIEX column at a flow rate of 3.0 ml/min. Following a washing step using, for instance, 20 mM Tris, pH 6.5, 5 mM DTT to remove unbound material, protein can be eluted with a single step of 8 column volumes of the following buffer (20 mM Tris, pH 6.5, 5 mM DTT, 1.0 M NaCl) The flow-through from the AIEX run can be loaded back onto the Q Sepharose column and eluted as described above to increase the yield. This two step technique (size exclusion followed by anion-exchange chromatography) effectively separates PCV-2 ORF2 antigen from most of the other protein components of the culture harvest.

Thus according to a preferred embodiment the present aspect of the invention provides a method of producing a PCV-2 antigenic composition comprising the steps of i) obtaining a first liquid containing a PCV-2 antigen, ii) removing at least a portion of the first liquid from the PCV-2 antigen and iii) purifying the harvest of step ii) comprising the PCV-2 antigen, by a two-step chromatography. Preferably the first chromatography step is different from the second step. If the first step is a size exclusion (gel filtration) chromatography, the second step can be ion-exchange chromatography, preferably anion-exchange chromatography (AIEX). Preferably, in any of the methods described above, which include one or more further purification steps to obtain a purified PCV-2 antigen, preferably a PCV-2 ORF-2 protein, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, and even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. In preferred forms, the method of producing a PCV-2 antigenic composition described above further comprises the steps of i) obtaining a PCV-2 antigen in a first liquid wherein the PCV-2 antigen is obtained via a viral vector, preferably a recombinant baculovirus viral vector, containing and expressing the PCV-2 antigen, preferably, PCV-2 ORF-2, and wherein the PCV-2 antigen comprises the ORF-2 protein of PCV-2, more preferably recombinant ORF-2 protein of PCV-2, and still more preferably virus like particles of ORF-2 protein; ii) removing at least a portion of the first liquid from the PCV-2 antigen; iii) inactivating the recombinant baculovirus viral vector with a DNA inactivating agent, preferably in the presence of about 1 to about 20 mM of binary ethylenimine; iv) adding an amount of a neutralizing agent that neutralizes the inactivating agent, the amount of neutralizing agent being equivalent to the amount of the inactivating agent, wherein the neutralizing agent preferably comprises a sodium thiosulfate solution preferably concentrated to a final concentration of about 1 to about 20 mM and wherein the inactivating agent preferably comprises BEI; and v) admixing the PCV-2 antigen obtained in step iv) with a further component selected from the group consisting of pharmaceutically acceptable carriers, adjuvants, diluents, excipients, and combinations thereof. The further purification, preferably, the two step purification strategy including the pre-filtration step results in an immunogenic composition having purity grade of more than 80% (w/w), preferably of more than 85% (w/w), even more preferred of more than 90% (w/w), most preferred of more than 95% (w/w) with reference to the total amount of protein included in the immunogenic composition prior to the mixture with any adjuvant.

The PCV-2 antigenic composition produced by the method described herein causes a loss of less than 1 log TCID₅₀ of a live virus or less than 1 log CFU per ml of a live bacterium, when the live virus or live bacterium is mixed with the PCV-2 antigenic composition and incubated for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, most preferably for more than 2 years. More preferably, the PCV-2 antigenic composition produced by the method described herein causes a loss of less than 0.9 log TCID₅₀ per ml of a live virus or less than 0.9 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, most preferably for more than 2 years. Even more preferably, the PCV-2 antigenic composition produced by the method described herein causes a loss of less than 0.7 log TCID₅₀ per ml of a live virus or less than 0.7 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, most preferably for more than 2 years. Still more preferably, the PCV-2 antigenic composition produced by steps by the method described herein causes a loss of less than 0.5 log TCID₅₀ per ml of a live virus or less than 0.5 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, most preferably for more than 2 years. Even more preferably, the PCV-2 antigenic composition produced by the method described herein causes a loss of less than 0.3 log TCID₅₀ per ml of a live virus or less than 0.3 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, most preferably for more than 2 years. The live virus can be any live virus, but preferably the live virus is the PRRS virus, preferably the PRRS virus having the ATCC accession number VR 2332. The live bacterium can be any bacterium, but is preferably the Mycoplasma hyopneumonia bacterium, preferably the J-strain of Mycoplasma hyopneumonia. The TCID₅₀ per ml can be estimated by a standard in vitro titration assay which allows the estimation of the amount of a live virus. The CFU per ml can be determined also by a standard in vitro titration assay which allows the estimation of the amount of a live bacterium. The term “per ml” preferably refers to 1 ml of a fluid. Such purified PCV-2 antigen, does not only show reduced virucidal activity, as defined herein, it also shows an increased immunogenicity as compared to a non-purified PCV-2 antigen as defined herein, preferably such purified PCV-2 antigen increases the cellular and/or antibody mediated immune response by at least 10%, preferably by at least 20%, more preferably by at least 30%, even more preferably by at least 40%, even more preferably by at least 50%, even more preferably by at least 75%, most preferably by at least 100% as compared to the cellular and/or antibody mediated immune response elicited by a reference immunogenic composition comprising a non-purified PCV-2 antigen.

Thus according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, wherein the PCV-2 antigenic composition obtained after step ii) causes a loss of less than 1 log TCID₅₀—preferably per ml—, preferably less than 0.9 log TCID₅₀,—preferably per ml—, even more preferably less than 0.7 log TCID₅₀—preferably per ml—, even more preferably less than 0.5 log TCID₅₀—preferably per ml—, most preferably less than 0.3 log TCID₅₀—preferably per ml—of a live virus, preferably of a live PRRSV or less than 1 log CFU—preferably per ml—, preferably less than 0.9 log CFU—preferably per ml—, even more preferably less than 0.7 log CFU—preferably per ml—, even more preferably less than 0.5 log CFU—preferably per ml—, most preferably less than 0.3 log CFU—preferably per ml—of a live bacterium, preferably of Mycoplasma hyopneumoniae, when the live virus, preferably PRRSV or live bacterium, preferably Mycoplasma hyopneumoniae is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, most preferably for more than 2 years. Preferably, the portion of the first liquid is removed from the PCV-2 antigen by an exchange of a portion of the first liquid against a second liquid. The exchange is preferably done in such that it comprises the steps of a) adding the second liquid to the first liquid which contains the PCV-2 antigen and b) concentrating the PCV-2 antigen, preferably from 3× to 50×, even more preferably from 4× to 20×, even more preferably from 7× to 10× in comparison to the volume of the first liquid by removing a portion of the first and second liquids from the PCV-2 antigen. Preferably, the liquid addition step and concentration step are performed multiple times, preferably two times, even more preferably three times. In such case, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultra-filtration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. When the PCV-2 antigen is obtained via a viral vector, preferably a recombinant baculovirus viral vector, containing and expressing the PCV-2 antigen, preferably, PCV-2 ORF-2, the process further comprises iii) inactivating the recombinant baculovirus viral vector with a DNA inactivating agent, preferably in the presence of about 1 to about 20 mM of binary ethylenimine; iv) adding an amount of a neutralizing agent that neutralizes the inactivating agent, the amount of neutralizing agent being equivalent to the amount of the inactivating agent, wherein the neutralizing agent preferably comprises a sodium thiosulfate solution preferably concentrated to a final concentration of about 1 to about 20 mM and wherein the inactivating agent preferably comprises BEI. Preferably, the inactivating and neutralization steps are performed after at least a portion of the first liquid is removed from the PCV-2 antigen, more preferably after the PCV-2 antigen is harvested. Even more preferably, the inactivating and neutralization steps are performed after the portion of the first liquid is removed from the PCV-2 antigen by an exchange of a portion of the first liquid against a second liquid. When the exchange of a portion of the first liquid against a second liquid is done such that it comprises the steps of a) adding the second liquid to the first liquid which contains the PCV-2 antigen and b) concentrating the PCV-2 antigen, preferably from 3× to 50×, even more preferably from 4× to 20×, even more preferably from 7× to 10× in comparison to the volume of the first liquid by removing a portion of the first and second liquids from the PCV-2 antigen, the inactivating and neutralization steps are done after the concentration step. When the liquid addition step and concentration step are performed multiple times, preferably two times, and even more preferably three times, such inactivation and neutralization steps are performed after the last liquid addition step and concentration step. When the concentration step is done by filtration—preferably by dia- and/or ultrafiltration, utilizing a filter, preferably containing a semi-permeable membrane, the inactivation and neutralization steps are performed after the filtration step described above, preferably utilizing a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. Preferably, further purification to obtain a purified PCV-2 antigen as defined herein, can be achieved by performing further purification step comprising iii) purifying the harvest of step ii) comprising the PCV-2 antigen, which is obtained after the removal of a portion of the first liquid, by a chromatography a step. In order to obtain a higher purity grade a second chromatography step can be done, which however is different from the first one. For instance if the first purification step/chromatography step is size exclusion (gel filtration) the second should different from that e.g. an affinity chromatography, ion exchange chromatography, etc. Preferably, if the first step to purify PCV-2 antigen, preferably to purify PCV-2 ORF2 antigen is a size exclusion (gel filtration) chromatography, the second step can be ion-exchange chromatography, preferably anion-exchange chromatography (AIEX). A preferred anion-exchange chromatography matrix for the purification of PCV-2 antigen, preferably the PCV-2 ORF2 antigen is Q Sepharose. In a small scale of about 50 ml, use of 5 ml HiTrap Q Sepharose HP columns are most preferred. The anion exchange chromatography can be conducted, for instance, as described in Example 3. Briefly, about 50 ml of the void volume fraction pool from the size exclusion chromatography step can be loaded onto the AIEX column at a flow rate of 3.0 ml/min. Following a washing step using, for instance, 20 mM Tris, pH 6.5, 5 mM DTT to remove unbound material, protein can be eluted with a single step of 8 column volumes of the following buffer (20 mM Tris, pH 6.5, 5 mM DTT, 1.0 M NaCl) The flow-through from the AIEX run can be loaded back onto the Q Sepharose column and eluted as described above to increase the yield. This two step technique (size exclusion followed by anion-exchange chromatography) effectively separates PCV-2 ORF2 antigen from most of the other protein components of the culture harvest.

The PCV-2 antigenic composition obtained according to the method described above, or the PCV-2 antigen used in step i) of the method described above, can be combined with at least one additional antigen, preferably a viral or bacterial antigen, and even more preferably, a viral or bacterial antigen from at least one other disease-causing organism in swine. The additional antigen can be any one of those disclosed in the international patent aspect of the invention WO2007/094893 (the contents and teachings of which are hereby incorporated by reference). Briefly, the additional antigens can be antigens of any other disease-causing organisms of swine. Preferably the “another disease-causing organisms” of swine are selected from the group consisting of: Actinobacillus pleuropneumonia (1); Adenovirus (2); Alphavirus such as Eastern equine encephalomyelitis viruses (3); Bordetella bronchiseptica (4); Brachyspira spp. (5), preferably B. hyodyentheriae (6); B. piosicoli (7), Brucella suis, preferably biovars 1, 2, and 3 (8); Classical swine fever virus (9); Clostridium spp. (10), preferably Cl. difficile (11), Cl. perfringens types A, B, and C (12), Cl. novyi (13), Cl. septicum (14), Cl. tetani (15); Coronavirus (16), preferably Porcine Respiratory Corona virus (17) or Porcine Epidemic Diarrhea Virus (18); Eperythrozoonosis suis (19); Erysipelothrix rhsiopathiae (20); Escherichia coli (21); Haemophilus parasuis, preferably subtypes 1, 7 and 14 (22) Hemagglutinating encephalomyelitis virus (23); Japanese Encephalitis Virus (24); Lawsonia intracellularis (25) Leptospira spp. (26), preferably Leptospira australis (27); Leptospira canicola (28); Leptospira grippotyphosa (29); Leptospira icterohaemorrhagicae (30); and Leptospira interrogans (31); Leptospira pomona (32); Leptospira tarassovi (33); Mycobacterium spp. (34) preferably M. avium (35), M. intracellulare (36) and M. bovis (37); Mycoplasma hyopneumoniae (38); Pasteurella multocida (39); Porcine cytomegalovirus (40); Porcine Parvovirus (41); Porcine Reproductive and Respiratory Syndrome Virus (42); Pseudorabies virus (43); Rotavirus (44); Salmonella spp. (45), preferably S. thyhimurium (46) and S. choleraesuis (47); Staph. hyicus (48); Staphylococcus spp. (49) preferably Streptococcus spp. (50), preferably Strep. suis (51); Swine herpes virus (52); Swine Influenza Virus (53); Swine pox virus (54); Swine pox virus (55); Vesicular stomatitis virus (56); Virus of vesicular exanthema of swine (57); Leptospira Hardjo (58); and/or Mycoplasma hyosynoviae (59).

Thus, according to a further aspect of the present aspect of the invention, the present aspect of the invention provides a method of producing a PCV-2 antigenic composition comprising the steps of i) obtaining a PCV-2 antigen in a first liquid; ii) removing at least a portion of the first liquid from the PCV-2 antigen; and combining the PCV-2 antigen with at least one additional antigen, preferably a viral or bacterial antigen, and more preferably a viral or bacterial antigen from at least one other disease-causing organism in swine. Preferably, the PCV-2 antigen comprises the ORF-2 protein of PCV-2, more preferably recombinant ORF-2 protein of PCV-2, and still more preferably virus like particles of ORF-2 protein. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, and even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. Further purification to obtain a purified PCV-2 antigen can be done as described above.

In preferred forms, the method of producing a PCV-2 antigenic composition described above further comprises the steps of i) obtaining a PCV-2 antigen in a first liquid wherein the PCV-2 antigen is obtained via a viral vector, preferably a recombinant baculovirus viral vector, containing and expressing the PCV-2 antigen, preferably, PCV-2 ORF-2, and wherein the PCV-2 antigen comprises the ORF-2 protein of PCV-2, more preferably recombinant ORF-2 protein of PCV-2, and still more preferably virus like particles of ORF-2 protein; ii) removing at least a portion of the first liquid from the PCV-2 antigen; iii) inactivating the recombinant baculovirus viral vector with a DNA inactivating agent, preferably in the presence of about 1 to about 20 mM of binary ethylenimine; iv) adding an amount of a neutralizing agent that neutralizes the inactivating agent, the amount of neutralizing agent being equivalent to the amount of the inactivating agent, wherein the neutralizing agent preferably comprises a sodium thiosulfate solution preferably concentrated to a final concentration of about 1 to about 20 mM and wherein the inactivating agent preferably comprises BEI; and v) admixing the PCV-2 antigen obtained in step iv) with a further component selected from the group consisting of pharmaceutically acceptable carriers, adjuvants, diluents, excipients, and combinations thereof.

In a further aspect of the method, the at least one additional antigen is a viral antigen, preferably an antigen from Porcine Reproductive and Respiratory Syndrome Virus. Even more preferably, the Porcine Reproductive and Respiratory Syndrome Virus antigen comprises a live virus, and still more preferably a modified live virus, even more preferably a modified live attenuated virus. Still more preferably, the modified live Porcine Reproductive and Respiratory Syndrome Virus antigen comprises a modified live virus strain of ATCC Accession Number VR 2332, and still more preferably comprises INGELVAC® PRRS MLV. Thus, according to a further aspect, the present aspect of the invention provides a method of producing a PCV-2 antigenic composition comprising the steps of i) obtaining a first liquid containing a PCV-2 antigen, ii) removing at least a portion of the first liquid from the PCV-2 antigen, and combining the PCV-2 antigen with an antigen from Porcine Reproductive and Respiratory Syndrome Virus. Preferably, the Porcine Reproductive and Respiratory Syndrome Virus antigen comprises a live virus, still more preferably a modified live virus, and even more preferably a modified live attenuated virus. Still more preferably, the modified live Porcine Reproductive and Respiratory Syndrome Virus antigen comprises a modified live virus strain of ATCC Accession Number VR 2332, and still more preferably comprises INGELVAC® PRRS MLV. Preferably the PCV-2 antigen comprises the ORF-2 protein of PCV-2, more preferably recombinant ORF-2 protein of PCV-2, and still more preferably virus like particles of ORF-2 protein. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, even more preferably three times. In such case, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. Further purification to obtain a purified PCV-2 antigen can be done as described above.

In a further aspect of the present application, the at least one additional antigen is a bacterial antigen, preferably Mycoplasma hyopneumoniae. Preferably the Mycoplasma hyopneumoniae antigen is a bacterin, and more preferably, the Mycoplasma hyopneumoniae bacterin is INGELVAC® MYCOFLEX. Thus, according to a further aspect, the present application provides a method of producing a PCV-2 antigenic composition comprising the steps of i) obtaining a first liquid containing a PCV-2 antigen, ii) removing at least a portion of the first liquid from the PCV-2 antigen, and combining the PCV-2 antigen with a bacterial antigen, preferably Mycoplasma hyopneumoniae. Preferably the Mycoplasma hyopneumoniae antigen is a bacterin, and more preferably, the Mycoplasma hyopneumoniae bacterin is INGELVAC® MYCOFLEX. Preferably the PCV-2 antigen comprises the ORF-2 protein of PCV-2, more preferably recombinant ORF-2 protein of PCV-2, and still more preferably virus like particles of ORF-2 protein. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, and even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. Further purification to obtain a purified PCV-2 antigen can be done as described above.

In a further aspect of the present application, the at least one additional antigen includes a viral antigen, preferably a Porcine Reproductive and Respiratory Syndrome Virus antigen, as described above, and a bacterial antigen, preferably a Mycoplasma hyopneumoniae antigen, as described above. Preferably, the Porcine Reproductive and Respiratory Syndrome Virus antigen comprises a live virus, more preferably a modified live virus, and still more preferably, comprises a modified live virus strain of ATCC Accession Number VR 2332, and still more preferably comprises INGELVAC® PRRS MLV. Preferably, the Mycoplasma hyopneumoniae antigen is a bacterin, and more preferably, the Mycoplasma hyopneumoniae bacterin is INGELVAC® MYCOFLEX Thus, according to a further aspect, the present application provides a method of producing a PCV-2 antigenic composition comprising the steps of i) obtaining a first liquid containing a PCV-2 antigen, ii) removing at least a portion of the first liquid from the PCV-2 antigen, and combining the PCV-2 antigen with a viral antigen, preferably a Porcine Reproductive and Respiratory Syndrome Virus antigen, as described above, and a bacterial antigen, preferably a Mycoplasma hyopneumoniae antigen, as described above. Preferably, the Porcine Reproductive and Respiratory Syndrome Virus antigen comprises a live virus, more preferably a modified live virus, and still more preferably, comprises a modified live virus strain of ATCC Accession Number VR 2332, and still more preferably comprises INGELVAC® PRRS MLV. Preferably, the Mycoplasma hyopneumoniae antigen is a bacterin, and more preferably, the Mycoplasma hyopneumoniae bacterin is INGELVAC® MYCOFLEX. Preferably the PCV-2 antigen comprises the ORF-2 protein of PCV-2, more preferably recombinant ORF-2 protein of PCV-2, and still more preferably virus like particles of ORF-2 protein. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, and even more preferably three times. In such cases, not only the first liquid is removed, but also a mixture of the first and second liquid. Preferably the liquid addition step is performed substantially simultaneously or sequentially as described above. When the concentrating step and liquid addition step are performed sequentially, the order of the steps does not matter. Moreover, the concentration step is preferably done by filtration—preferably by dia- and/or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. Further purification to obtain a purified PCV-2 antigen can be done as described above.

The present aspect of the invention does not only provide methods of producing PCV-2 antigenic compositions, it also relates to a PCV-2 antigenic composition. Thus, according to a further aspect the present patent application further provides a PCV-2 antigenic composition characterized in such that the PCV-2 antigenic composition causes a loss of less than 1 log TCID₅₀ of a live virus or less than 1 log CFU per ml of a live bacterium, when the live virus or live bacterium is mixed with the PCV-2 antigenic composition and incubated for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, and most preferably for more than 2 years. More preferably, the PCV-2 antigenic composition produced by the method described herein causes a loss of a live virus or less than 0.9 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, and most preferably for more than 2 years. Even more preferably, the PCV-2 antigenic composition causes a loss of less than 0.7 log TCID₅₀ per ml of a live virus or less than 0.7 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, and most preferably for more than 2 years. Still more preferably, the PCV-2 antigenic composition causes a loss of less than 0.5 log TCID₅₀ per ml of a live virus or less than 0.5 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, and most preferably for more than 2 years. Even more preferably, the PCV-2 antigenic composition causes a loss of less than 0.3 log TCID₅₀ per ml of a live virus or less than 0.3 log CFU per ml of a live bacterium, when the live virus or live bacterium is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, and most preferably for more than 2 years. The live virus can be any live virus, but preferably the live virus is the PRRS virus, preferably the PRRS virus having the ATCC accession number VR 2332. The live bacterium can be any bacterium, but is preferably the Mycoplasma hyopneumonia bacterium, preferably the J-strain of Mycoplasma hyopneumonia. The TCID₅₀ per ml can be estimated by the estimation of the amount of a live virus. The CFU per ml can be determined also by a standard in vitro titration assay which allows the estimation of the amount of a live bacterium. The term “per ml” preferably refers to 1 ml of a fluid.

In a further aspect, the PCV-2 antigenic composition described above comprises a further component selected from the group consisting of pharmaceutically acceptable carriers, adjuvants, diluents, excipients, and combinations thereof. Preferably, the further component is an adjuvant, even more preferably wherein the adjuvant is a polymer of acrylic or methacrylic acid, and still more preferably wherein the adjuvant is Carbomer. Preferably, the adjuvant is added in an amount of about 100 μg to about 10 mg per dose. Even more preferably the adjuvant is added in an amount of about 100 μg to about 10 mg per dose. Still more preferably the adjuvant is added in an amount of about 500 μg to about 5 mg per dose. Still more preferably the adjuvant is added in an amount of about 750 μg to about 2.5 mg per dose. Most preferably the adjuvant is added in an amount of about 1 mg per dose.

The present aspect of the invention does not only provide methods of producing PCV-2 antigenic compositions and/or the PCV-2 antigenic compositions as defined above, it also relates to a PCV-2 antigenic composition that is obtainable by any of the methods described herein. Thus, in a further aspect the present aspect of the invention relates to a PCV-2 antigenic composition that is obtained by a method comprising the steps of i) obtaining a first liquid containing a PCV-2 antigen, ii) removing at least a portion of the first liquid from the PCV-2 antigen. Preferably the PCV-2 antigen is used as or in the PCV-2 antigenic composition. The term “a PCV-2 antigenic composition obtained by a method provided herein” also means that the PCV-2 antigenic composition is obtainable by a method provided herein. According to a further aspect, the present aspect of the invention also relates to the PCV-2 antigenic composition that is obtained by removing the portion of the first liquid from the PCV-2 antigen by an exchange of the portion of the first liquid against a second liquid, wherein the second liquid is different from the first liquid. Thus according to a further aspect, the present application relates to a PCV-2 antigenic composition obtained by a method comprising the steps of i) obtaining a first liquid containing a PCV-2 antigen, ii) removing at least a portion of the first liquid from the PCV-2 antigen, wherein the portion of the first liquid is removed from the PCV-2 antigen by an exchange of the portion of the first liquid against a second liquid, wherein the second liquid is different from the first liquid. Preferably the exchange of the portion of the first liquid with the second liquid comprises the steps of a) adding the second liquid to the first liquid which contains the PCV-2 antigen and b) concentrating the PCV-2 antigen by removing a portion of the first and second liquids from the PCV-2 antigen.

According to a further aspect, the PCV-2 antigenic composition is preferably obtained by a method wherein the portion of the first liquid is removed from the PCV-2 antigen by a filtration step utilizing a filter. However, any other methods known to a person skilled in the art can be used to remove the portion of the first and second fluid from the PCV-2 antigen, for instance centrifugation and/or chromatography. However, filtration is most preferred. Preferred filtration methods to remove the portion of the first fluid comprise ultra- and/or dia-filtration. The concentrating step and the liquid addition step of the method as described herein can be performed substantially simultaneously or alternatively, the concentrating step and the liquid addition step are performed sequentially. Thus according to a further aspect, the present aspect of the invention relates to a PCV-2 antigenic composition obtained by a method comprising the steps of i) obtaining a first liquid containing a PCV-2 antigen, ii) removing at least a portion of the first liquid from the PCV-2 antigen, wherein the portion of the first liquid is removed from the PCV-2 antigen by an exchange of the portion of the first liquid against a second liquid, wherein the second liquid is different from the first liquid. Preferably the exchange of the portion of the first liquid with the second liquid comprises the steps of a) adding the second liquid to the first liquid which contains the PCV-2 antigen and b) concentrating the PCV-2 antigen by removing a portion of the first and second liquids from the PCV-2 antigen, wherein the liquid addition step is performed substantially simultaneously or sequentially. When the concentrating step and liquid addition step are performed sequentially, the order of the steps does not matter. For example, in a further aspect, the liquid addition step occurs prior to the concentrating step and in an alternative aspect, the concentrating step occurs prior to the liquid addition step.

In a further aspect, the present aspect of the invention relates to a PCV-2 antigenic composition that can be obtained using a method described herein, wherein the liquid addition step and the concentrating step, regardless of the order in which they are performed, can be performed multiple times. For example, each of these respective steps can be performed at least two, at least three, at least four, at least five, at least 10, up to as many times as desired. In one aspect, the concentrating step and the liquid addition step are each performed at least two times. In another aspect, the concentrating step and the liquid addition step are each performed at least three times.

In a further aspect of the present application, the PCV-2 antigenic composition of the present aspect of the invention is obtained as described above, wherein filtration is the preferred method to remove a portion of the first liquid, or in case of multiple removing steps as described above, a portion of the mixture of the first and the second fluid from the PCV-2 antigen. The filter can be any conventional filter in the art. Preferably, the filter includes a semi-permeable membrane. In a further preferred form, the semi-permeable membrane has an average pore size that is smaller than the PCV-2 antigen to thereby prevent passage of at least 90% of the PCV-2 antigen through the semi-permeable membrane pores and withhold the PCV-2 antigen by the filter. In a further aspect, the filter has an average pore size which prevents passage of at least 90% of proteins of 50 kDa to 500 kDa in size, more preferably, the filter has an average pore size which prevents passage of at least 90% of proteins of 75 kDa to 400 kDa in size, and most preferably, the filter has an average pore size which prevents passage of at least 90% of proteins of 100 kDa to 300 kDa in size. This pore size is preferred, when the PCV-2 antigen is produced as whole virus or as virus like particles. In a still further aspect, the semi-permeable membrane includes a material selected from the group consisting of polysulfone, polyethersulfone, and regenerated cellulose. However, any other material can be used, which allows removing of a portion of the first fluid, and in case of a multiple process step, removing of a mixture of the first and the second fluid from the PCV-2 antigen. In a further aspect, the filter is selected from the group consisting of a hollow fiber membrane ultra filtration cartridge, flat sheets, or a cassette, with a hollow fiber membrane ultra filtration cartridge being particularly preferred.

Thus, according to a further aspect, the present aspect of the invention relates to a PCV-2 antigenic composition that is obtained using the methods as described above, wherein the filter preferably is or comprises a semi-permeable membrane. Preferably, the semi-permeable membrane has an average pore size that is smaller than the PCV-2 antigen and prevents passage of at least 90% of the PCV-2 antigen through the semi-permeable membrane pores. Preferably the average pore size of the semi-permeable membrane prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. As described above, the removing step in general includes the exchange of the portion of the first fluid against a portion of the second fluid comprises the steps of a) adding the second liquid to the first liquid which contains the PCV-2 antigen and b) concentrating the PCV-2 antigen by removing a portion of the first and second liquids from the PCV-2 antigen, wherein the liquid addition step and concentration step are performed multiple times, for instance, two times, three times, 5 times, 10 times, etc. Preferably, the liquid addition step and concentration step are performed two times, most preferably three times.

The concentration step of the method provided herein to obtain the PCV-2 antigenic composition is performed such that the PCV-2 antigen is concentrated from 3× to 50× in comparison to the volume of the first liquid. More preferably, the concentrating step is done in such that the PCV-2 antigen is concentrated 4× to 20× in comparison to the volume of the first liquid. Most preferably, concentration step is done in such that the PCV-2 antigen is concentrated from 7× to 10× in comparison to the volume of the first liquid. Thus according to a further aspect, the present aspect of the invention relates to a PCV-2 antigenic composition obtained by a method described above, wherein the PCV-2 antigen is concentrated from 3× to 50×, preferably from 4× to 20×, and even more preferably from 7× to 10× in comparison to the volume of the first liquid. Preferably, the portion of the first fluid is removed from the PCV-2 antigen by an exchange of the portion of the first liquid against a second liquid comprising the steps of a) adding the second liquid to the first liquid which contains the PCV-2 antigen and b) concentrating the PCV-2 antigen from 3× to 50×, preferably from 4× to 20×, and even more preferably from 7× to 10× in comparison to the volume of the first liquid by removing a portion of the first and second liquids from the PCV-2 antigen. Preferably the liquid addition step is performed substantially simultaneously or sequentially with the concentrating step. When the concentrating step and liquid addition step are performed sequentially, the order of the steps does not matter. Moreover, the concentration step is preferably done by filtration—preferably dia- and/or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores. Preferably the average pore size of the semi-permeable membrane prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

Preferably, further purification to obtain PCV-2 antigenic composition comprising a purified PCV-2 antigen as defined herein, can be achieved by performing further purification step comprising iii) purifying the harvest of step ii) comprising the PCV-2 antigen (of any methods described herein), which is obtained after the removal of a portion of the first liquid, by a chromatography a step. In order to obtain a higher purity grade a second chromatography step can be done, which however is different from the first one. For instance if the first purification step/chromatography step is size exclusion (gel filtration) the second should different from that e.g. an affinity chromatography, ion exchange chromatography, etc. Preferably, if the first step to purify PCV-2 antigen, preferably to purify PCV-2 ORF2 antigen is a size exclusion (gel filtration) chromatography, the second step can be ion-exchange chromatography, preferably anion-exchange chromatography (AIEX). A preferred anion-exchange chromatography matrix for the purification of PCV-2 antigen, preferably the PCV-2 ORF2 antigen is Q Sepharose. In a small scale of about 50 ml, use of 5 ml HiTrap Q Sepharose HP columns are most preferred. The anion exchange chromatography can be conducted, for instance, as described in Example 3. Briefly, about 50 ml of the void volume fraction pool from the size exclusion chromatography step can be loaded onto the AIEX column at a flow rate of 3.0 ml/min. Following a washing step using, for instance, 20 mM Tris, pH 6.5, 5 mM DTT to remove unbound material, protein can be eluted with a single step of 8 column volumes of the following buffer (20 mM Tris, pH 6.5, 5 mM DTT, 1.0 M NaCl) The flow-through from the AIEX run can be loaded back onto the Q Sepharose column and eluted as described above to increase the yield. This two step technique (size exclusion followed by anion-exchange chromatography) effectively separates PCV-2 ORF2 antigen from most of the other protein components of the culture harvest.

In a further aspect, the virucidal activity of the PCV-2 antigenic composition produced by the methods described herein is reduced by at least 10% as compared to the liquid that has not undergone the method. More preferably, the virucidal activity of the PCV-2 antigenic composition is reduced by at least 50% as compared to the first liquid that has not undergone the method. Still more preferably, the virucidal activity of the PCV-2 antigenic composition is reduced by at least 70% as compared to the first liquid that has not undergone the method.

Thus according to a further aspect, the present aspect of the invention relates to PCV-2 antigenic composition obtained by a method comprising the steps of i) obtaining a first liquid containing a PCV-2 antigen, ii) removing at least a portion of the first liquid from the PCV-2 antigen, wherein the virucidal activity—preferably in respect to PRRS virus—of the PCV-2 antigenic composition obtained after step ii) is reduced by at least 10%, preferably at least 50%, more preferably at least 70%, even more preferably at least 90% as compared to that of the first liquid. Preferably, the portion of the first liquid having virucidal activity is removed from the PCV-2 antigen by an exchange of a portion of the first liquid against a second liquid. The exchange is preferably done in such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably the liquid addition step is performed substantially simultaneously or sequentially with the concentrating step as described above. When the concentrating step and liquid addition step are performed sequentially, the order of the steps does not matter. Moreover, the concentration step is preferably done by filtration—preferably by dia- and/or ultra-filtration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles. Further purification to obtain a purified PCV-2 antigen can be done as described above.

According to a further aspect, the present aspect of the invention relates to a PCV-2 antigenic composition obtained by a method described herein, wherein the PCV-2 antigenic composition causes a loss of less than 1 log TCID₅₀—preferably per ml—, preferably less than 0.9 log TCID₅₀,—preferably per ml—, even more preferably less than 0.7 log TCID₅₀—preferably per ml—, even more preferably less than 0.5 log TCID₅₀—preferably per ml—, most preferably less than 0.3 log TCID₅₀—preferably per ml—of a live virus, preferably of a live PRRSV or less than 1 log CFU—preferably per ml—, preferably less than 0.9 log CFU—preferably per ml—, even more preferably less than 0.7 log CFU—preferably per ml—, even more preferably less than 0.5 log CFU—preferably per ml—, most preferably less than 0.3 log CFU—preferably per ml—of a live bacterium, preferably of Mycoplasma hyopneumoniae, when the live virus, preferably PRRSV or live bacterium, preferably Mycoplasma hyopneumoniae is mixed and incubated with the PCV-2 antigenic composition for 2 or more hours, preferably for more than 4 hours, even more preferably for more than 12 hours, even more preferably for more than 24 hours, even more preferably for more than 2 days, even more preferably for more than 4 days, even more preferably for more than 7 days, even more preferably for more than 2 weeks, even more preferably for more than 4 weeks, even more preferably for more than 2 months, even more preferably for more than 3 months, even more preferably for more than 4 months, even more preferably for more than 6 months, even more preferably for more than 9 months, even more preferably for more than 12 months, even more preferably for more than 18 months, and most preferably for more than 2 years. The live virus can be any live virus, but preferably the live virus is the PRRS virus, preferably the PRRS virus having the ATCC accession number VR 2332. The live bacterium can be any bacterium, but is preferably the Mycoplasma hyopneumoniae bacterium, preferably the J-strain of Mycoplasma hyopneumoniae. The TCID₅₀ per ml can be estimated by a standard in vitro titration assay which allows the estimation of the amount of a live virus. The CFU per ml can be determined also by a standard in vitro titration assay which allows the estimation of the amount of a live bacterium. The term “per ml” preferably refers to 1 ml of a fluid.

In a further aspect, the present aspect of the invention relates to a PCV-2 antigenic composition that is obtained by a method described above, further comprising the step of harvesting the PCV-2 antigen remaining after step ii). This harvesting can be done in any conventional manner. In a particularly preferred manner of harvesting, the portion of the first liquid is removed from the PCV-2 antigen via a filtration step and the PCV-2 antigen is recovered or harvested from the filter retard.

In a further aspect, the PCV-2 antigenic composition obtained by any of the methods described herein is admixed with a further component selected from the group consisting of pharmaceutically acceptable carriers, adjuvants, diluents, excipients, and combinations thereof. Preferably, the further component is an adjuvant, even more preferably wherein the adjuvant is a polymer of acrylic or methacrylic acid, and still more preferably wherein the adjuvant is Carbomer.

Thus, according to a further aspect, the present aspect of the invention provides a PCV-2 antigenic composition obtained by a method described above, further comprising the step of admixing the PCV-2 antigen obtained by the method described herein with a further component selected from the group consisting of pharmaceutically acceptable carriers, adjuvants, diluents, excipients, and combinations thereof. Preferably the further component is an adjuvant, even more preferably wherein the adjuvant is a polymer of acrylic or methacrylic acid, and still more preferably wherein the adjuvant is Carbomer. Preferably, the adjuvant is added in an amount of about 100 μg to about 10 mg per dose. Even more preferably the adjuvant is added in an amount of about 100 μg to about 10 mg per dose. Still more preferably the adjuvant is added in an amount of about 500 μg to about 5 mg per dose. Still more preferably the adjuvant is added in an amount of about 750 μg to about 2.5 mg per dose. Most preferably the adjuvant is added in an amount of about 1 mg per dose.

In a further aspect, the PCV-2 antigenic composition described above comprises the ORF-2 protein of PCV-2, more preferably recombinant ORF-2 protein of PCV-2, and still more preferably virus like particles of ORF-2 protein. Thus, according to a further aspect of the present application, the present aspect of the invention provides a PCV-2 antigenic composition obtained by a method described above, wherein the PCV-2 antigen comprises the ORF-2 protein of PCV-2, more preferably recombinant ORF-2 protein of PCV-2, and still more preferably virus like particles of ORF-2 protein.

As mentioned above, the PCV-2 antigen used in the method described herein can be obtained by any method known in the art. Preferably, the PCV-2 antigen is obtained via a viral vector, preferably a recombinant baculovirus viral vector, containing and expressing the PCV-2 antigen, preferably, PCV-2 ORF-2. In preferred forms, the PCV-2 antigen is obtained following the procedures described in WO2006/072065 (the teachings and content of which were previously incorporated by reference). Thus, according to a further aspect of the present application, the present aspect of the invention provides a PCV-2 antigenic composition obtained by a method described above, wherein the PCV-2 antigen is obtained via a viral vector, preferably a recombinant baculovirus viral vector, containing and expressing the PCV-2 antigen, preferably, PCV-2 ORF-2, and wherein the PCV-2 antigen comprises the ORF-2 protein of PCV-2, more preferably recombinant ORF-2 protein of PCV-2, and still more preferably virus like particles of ORF-2 protein.

In a further aspect of the present aspect of the invention, the PCV-2 antigenic composition is obtained by the method described above and further comprises the step of inactivating the recombinant baculovirus viral vector with a DNA inactivating agent, preferably in the presence of about 1 to about 20 mM of binary ethylenimine. In preferred forms, the method further comprises the step of adding an amount of an agent that neutralizes the DNA inactivating agent, the amount being equivalent to the amount of the DNA inactivating agent wherein the agent that neutralizes the DNA inactivating agent comprises a sodium thiosulfate solution concentrated to a final concentration of about 1 to about 20 mM and wherein the DNA inactivating agent is BEI. Preferably, the inactivating step is performed after at least a portion of the first liquid is removed from the PCV-2 antigen.

In a further aspect of the present aspect of the invention, the PCV-2 antigenic composition is obtained by the method described above further comprising the steps of admixing the PCV-2 antigen obtained after the inactivating and neutralizing steps. Thus, according to a further aspect, the present aspect of the invention provides a PCV-2 antigenic composition obtained by a method described above comprising the steps of i) obtaining a PCV-2 antigen in a first liquid; ii) removing at least a portion of the first liquid from the PCV-2 antigen; iii) inactivating the recombinant baculovirus viral vector with a DNA inactivating agent, preferably in the presence of about 1 to about 20 mM of binary ethylenimine; iv) adding an amount of a neutralizing agent that neutralizes the inactivating agent, the amount of neutralizing agent being equivalent to the amount of the inactivating agent, wherein the neutralizing agent preferably comprises a sodium thiosulfate solution preferably concentrated to a final concentration of about 1 to about 20 mM and wherein the inactivating agent preferably comprises BEI; and, preferably step v), comprising admixing the PCV-2 antigen obtained in step iv) with a further component selected from the group consisting of pharmaceutically acceptable carriers, adjuvants, diluents, excipients, and combinations thereof.

In a further aspect of the present application, the PCV-2 antigenic composition described above, preferably obtained by the methods described above, further comprises at least one additional antigen, preferably a viral or bacterial antigen, and more preferably a viral or bacterial antigen from at least one other disease-causing organism in swine. In a further aspect the at least one additional antigen is Porcine Reproductive and Respiratory Syndrome Virus. Even more preferably, the Porcine Reproductive and Respiratory Syndrome Virus antigen comprises a live virus, and still more preferably a modified live virus. Still more preferably, the modified live Porcine Reproductive and Respiratory Syndrome Virus antigen comprises a modified live virus strain of ATCC Accession Number VR 2332, and still more preferably comprises INGELVAC® PRRS MLV. In a further aspect of the present application, the at least one additional antigen is Mycoplasma hyopneumoniae. Preferably the Mycoplasma hyopneumoniae antigen is a bacterin, and more preferably, the Mycoplasma hyopneumoniae bacterin is INGELVAC® MYCOFLEX. In a further aspect of the present aspect of the invention, the PCV-2 antigenic composition described above, preferably obtained by the methods described above further comprises Porcine Reproductive and Respiratory Syndrome Virus antigen, preferably a modified live Porcine Reproductive and Respiratory Syndrome Virus, still more preferably, the Porcine Reproductive and Respiratory Syndrome Virus having the ATCC Accession Number VR 2332, or the Porcine Reproductive and Respiratory Syndrome Virus included in INGELVAC® PRRS MLV or INGELVAC® PRRS ATP. In a further aspect of the present aspect of the invention, the PCV-2 antigenic composition described above, preferably obtained by the methods described above further comprises Mycoplasma hyopneumoniae, preferably Mycoplasma hyopneumoniae bacterin, and more preferably INGELVAC® MYCOFLEX or the Mycoplasma hyopneumoniae bacterin included in INGELVAC® MYCOFLEX. In a further aspect, the PCV-2 antigenic composition described herein comprises a Porcine Reproductive and Respiratory Syndrome Virus, preferably any one of those described above and a Mycoplasma hyopneumoniae, preferably any one of those described above.

When the PCV-2 antigenic composition comprising the at least one additional antigen from at least one other disease-causing organism in swine as described above, preferably Porcine Reproductive and Respiratory Syndrome Virus and/or Mycoplasma hyopneumoniae antigen is obtained by a method described herein, the method comprises the steps of i) obtaining a PCV-2 antigen in a first liquid; ii) removing at least a portion of the first liquid from the PCV-2 antigen; and combining the PCV-2 antigen with at least one additional antigen, preferably a viral or bacterial antigen, and more preferably a viral or bacterial antigen from at least one other disease-causing organism in swine. Preferably, the PCV-2 antigen comprises the ORF-2 protein of PCV-2, more preferably recombinant ORF-2 protein of PCV-2, and still more preferably virus like particles of ORF-2 protein. Preferably, the portion of the first liquid is removed from the PCV-2 antigen by an exchange of a portion of the first liquid against a second liquid. The exchange is preferably done such that it comprises the steps of a) adding the second liquid to the first liquid which contains the PCV-2 antigen and b) concentrating the PCV-2 antigen, preferably from 3× to 50×, even more preferably from 4× to 20×, and even more preferably from 7× to 10× in comparison to the volume of the first liquid by removing a portion of the first and second liquids from the PCV-2 antigen. Preferably, the liquid addition step and concentration step are performed multiple times, preferably two times, even more preferably three times. In such cases, not only the first liquid is removed, but also a mixture of the first and second liquid. Preferably each liquid addition step is performed substantially simultaneously or sequentially as described above. When the concentrating step and liquid addition step are performed sequentially, the order of the steps does not matter. Moreover, the concentration step is preferably done by filtration—preferably by dia- or ultrafiltration, utilizing a filter, which preferably contains a semi-permeable membrane. The semi-permeable membrane preferably has an average pore size that is smaller than the PCV-2 antigen and prevents passage of at least 90% of the PCV-2 antigen through the semi-permeable membrane pores and withholds the PCV-2 antigen within the filter for harvesting or recovery. Preferably the average pore size of the semi-permeable membrane or of any other filter that is used herein, prevents passage of at least 90% of proteins of 50 kDa to 500 kDa in size, more preferably, at least 90% of proteins of 75 kDa to 400 kDa in size, and most preferably at least 90% of proteins of 100 kDa to 300 kDa in size. This pore size is preferred, when the PCV-2 antigen is produced as whole virus or as virus like particles.

The present aspect of the invention as defined above, provides new methods of producing a PCV-2 antigen and immunogenic compositions comprising a PCV-2 antigen, wherein the PCV-2 antigen shows a reduced virucidal activity and/or increased immunogenicity (each as defined herein), wherein the method comprises the steps of i) obtaining a first liquid containing a PCV-2 antigen, ii) removing at least a portion of the first liquid from the PCV-2 antigen. Moreover, the present aspect of the invention also provides a PCV-2 antigen as well as immunogenic compositions comprising such PCV-2 antigen showing a reduced virucidal activity and/or increased immunogenicity (each as defined herein). According to a further aspect, the PCV-2 antigen as well as the immunogenic compositions comprising a purified PCV-2 antigen showing a reduced virucidal activity and/or increased immunogenicity can alternatively be obtained by the following method (II). The purified PCV-2 antigen according to the aspect of the invention, preferably the purified PCV-2 ORF2 antigen, can be obtained by the purification of a PCV-2 virus preparation, in particular by the purification of the whole virus. Whole virus preparations are described for instance in WO 99/18214 or WO 03/049703. Moreover, purified PCV-2 antigen can also be obtained by the purification of a recombinant expressed PCV-2 antigen, preferably by the purification of a recombinant PCV-2 ORF2 antigen. Expression systems for the production of recombinant PCV-2 antigen, preferably for the production of recombinant PCV-2 ORF2 antigens are well known in the art and include, but not limited to, bacterial expression systems, yeast expression systems, insect cell or mammalian expression systems. Vectors and methods for making and/or using vectors (or recombinants) for the expression of the PCV-2 antigens are described in the application elsewhere.

Preferred cells are those susceptible for infection with an appropriate recombinant viral vector, containing a PCV-2 ORF2 DNA and expressing the PCV-2 ORF2 protein. Preferably the cells are insect cells, and more preferably, they include the insect cells sold under the trademark SF+ insect cells (Protein Sciences Corporation, Meriden, Conn.). Preferred cell cultures have a cell count between about 0.3-2.0×10⁶ cells/mL, more preferably from about 0.35-1.9×10⁶ cells/mL, still more preferably from about 0.4-1.8×10⁶ cells/mL, even more preferably from about 0.45-1.7×10⁶ cells/mL, and most preferably from about 0.5-1.5×10⁶ cells/mL.

Preferred viral vectors include baculovirus such as BaculoGold (BD Biosciences Pharmingen, San Diego, Calif.), in particular provided that the production cells are insect cells. Although the baculovirus expression system is preferred, it is understood by those of skill in the art that other expression systems, including those described above will work for purposes of the present aspect of the invention, namely the expression of PCV-2 ORF2 antigen.

Appropriate growth media will also be determinable by those of skill in the art with a preferred growth media being serum-free insect cell media such as Excell 420 (JRH Biosciences, Inc., Lenexa, Kans.) and the like.

The recombinant viral vector containing the PCV-2 ORF2 DNA sequences has a preferred multiplicity of infection (MOI) of between about 0.03-1.5, more preferably from about 0.05-1.3, still more preferably from about 0.09-1.1, and most preferably from about 0.1-1.0, when used for the infection of the susceptible cells. Preferably the MOIs mentioned above relates to one mL of cell culture fluid. Preferably, the method described herein comprises the infection of 0.35-1.9×10⁶ cells/mL, still more preferably of about 0.4-1.8×10⁶ cells/mL, even more preferably of about 0.45-1.7×10⁶ cells/mL, and most preferably of about 0.5-1.5×10⁶ cells/mL with a recombinant viral vector containing a PCV-2 ORF2 DNA and expressing the PCV-2 ORF2 antigen protein having a MOI (multiplicity of infection) of between about 0.03-1.5, more preferably from about 0.05-1.3, still more preferably from about 0.09-1.1, and most preferably from about 0.1-1.0.

The infected cells are then incubated over a period of up to ten days, more preferably from about two days to about ten days, still more preferably from about four days to about nine days, and most preferably from about five days to about eight days. Preferred incubation conditions include a temperature between about 22-32° C., more preferably from about 24-30° C., still more preferably from about 25-29° C., even more preferably from about 26-28° C., and most preferably about 27° C. Preferably, the SF+ cells are observed following inoculation for characteristic baculovirus-induced changes. Such observation may include monitoring cell density trends and the decrease in viability during the post-infection period. It was found that peak viral titer is observed 3-5 days after infection and peak PCV-2 ORF2 antigen production in cells is obtained between days 5 and 8 post infection and/or when cell viability decreases to less than 10%.

The PCV-2 ORF2 antigen can be purified from the harvest by standard methods known to a person skilled in the art, for example by those described in Protein purification methods—a practical approach (E. L. V. Harris and S. Angal, eds., IRL Press at Oxford University Press). Those methods include, but are not limited to, separation by centrifugation and/or filtration, precipitation, size exclusion (gel filtration) chromatography, affinity chromatography, metal chelate chromatography, ion-exchange chromatography covalent chromatography, hydrophobic interaction chromatography, etc.

The recovery process of the PCV-2 antigen, preferably the PCV-2 ORF2 antigen, preferably begins with the separation of cell debris from the expressed PCV-2 ORF2 antigen via a separation step. Preferred separation steps include filtration, centrifugation at speeds up to about 20,000×g, continuous flow centrifugation, chromatographic separation using ion exchange or gel filtration, and conventional immunoaffinity methods. Those methods are known to persons skilled in the art for example by (E. L. V. Harris and S. Angel (eds.), Protein purification methods—a practical approach, IRL Press Oxford 1995). The most preferred separation methods include centrifugation at speeds up to about 20,000×g and filtration. Preferred filtration methods include dead-end microfiltration and tangential flow (or cross flow) filtration including hollow fiber filtration dead-end micro filtration. Of these, dead-end microfiltration is preferred. Preferred pore sizes for dead-end microfiltration are between about 0.30-1.35 μm, more preferably between about 0.35-1.25 μm, still more preferably between about 0.40-1.1 μm, and most preferably between about 0.45-1.0 μm. It is believed that any conventional filtration membrane will work for purposes of the present aspect of the invention and polyethersulfone membranes are preferred. Any low weight nucleic acid species are removed during the filtration step.

Further purification of PCV-2 antigen, preferably of the PCV-2 ORF2 antigen can be achieved with chromatography procedures, preferably a two-step chromatography procedure. However it is also possible to start with the chromatography procedure in the event, the loading material does not comprise cell debris.

If the PCV-2 antigen is assembled to virus like particles (VLP), the first step is preferably a size exclusion (gel filtration) chromatography, which can be done, for instance, by using a Sephacryl 5300 matrix. In lab scale use of HiPrep 26/60 Sephacryl S300HR columns are most preferred. However, any other size exclusion chromatography matrices known to a person skilled in the art can be used, which allow the separation of the PCV-2 ORF2 VLPs from the culture filtrate or supernatant. Suitable matrices are described, for instance, in E. L. V. Harris and S. Angel (eds.), Protein purification methods—a practical approach, IRL Press Oxford 1995). The gel filtration chromatography can be conducted, for instance, by loading the column with the crude preparation comprising the PCV-2 antigen with a flow-rate of 1.0 ml/min and eluting the column with 1.5 column volume of a buffer comprising 20 mM Tris, pH 6.5, 5 mM DTT. However, the PCV-2 ORF2 antigen can also be purified by using affinity chromatography, for instance, via selective binding to an immobilized PCV-2 ORF2 specific antibody, or any other method known to a person skilled in the art.

Thus according to a preferred embodiment, the immunogenic composition comprising a purified PCV-2 antigen, preferably a purified PCV-2 ORF2 antigen, and the adjuvant, is obtainable by a process comprising the steps:
a) Expressing the PCV-2 antigen, prefer the PCV-2 ORF2 antigen in a host cell;
b) Harvesting the cell culture obtaining PCV-2 antigen, preferably the PCV-2 ORF2 antigen;
c) Purifying the harvest comprising the PCV-2 antigen, preferably the PCV-2 ORF2 antigen by size exclusion chromatography (gel filtration);
d) Admixing the purified PCV-2 antigen, preferably the PCV-2 ORF2 antigen with an adjuvant.

According to a preferred embodiment, the size exclusion chromatography is performed as described herein, preferably as described in Example 3. Preferably, the size exclusion results in an immunogenic composition having purity grade of more than 80% (w/w), preferably more than 90% (w/w) with reference to the total amount of protein included in the immunogenic composition prior to the mixture with the adjuvant. The purity grade can be estimated by Imperial Protein Stain (Pierce) staining after SDS PAGE via NuPAGE 10% Bis-Tris gels (Invitrogen) using the NuPAGE MOPS buffer system (Invitrogen).

In order to obtain a higher purity grade a second chromatography step can be done, which however is different from the first one. For instance if the first purification step/chromatography step is size exclusion (gel filtration) the second should different from that e.g. an affinity chromatography, ion exchange chromatography, etc.

Preferably, if the first step to purify PCV-2 antigen, preferably to purify PCV-2 ORF2 antigen is a size exclusion (gel filtration) chromatography, the second step can be ion-exchange chromatography, preferably anion-exchange chromatography (AIEX). A preferred anion-exchange chromatography matrix for the purification of PCV-2 antigen, preferably the PCV-2 ORF2 antigen is Q Sepharose. In a small scale of about 50 ml, use of 5 ml HiTrap Q Sepharose HP columns are most preferred. The anion exchange chromatography can be conducted, for instance, as described in Example 3. Briefly, about 50 ml of the void volume fraction pool from the size exclusion chromatography step can be loaded onto the AIEX column at a flow rate of 3.0 ml/min. Following a washing step using, for instance, 20 mM Tris, pH 6.5, 5 mM DTT to remove unbound material, protein can be eluted with a single step of 8 column volumes of the following buffer (20 mM Tris, pH 6.5, 5 mM DTT, 1.0 M NaCl) The flow-through from the AIEX run can be loaded back onto the Q Sepharose column and eluted as described above to increase the yield. This two step technique (size exclusion followed by anion-exchange chromatography) effectively separates PCV-2 ORF2 antigen from most of the other protein components of the culture harvest.

Thus according to a preferred embodiment, the immunogenic composition comprising a purified PCV-2 antigen, preferably the PCV-2 ORF2 antigen, and the adjuvant, is obtainable by a process comprising the steps:
a) Expressing the PCV-2 antigen, prefer the PCV-2 ORF2 antigen in a host cell;
b) Harvesting the cell culture obtaining PCV-2 antigen, preferably the PCV-2 ORF2 antigen;
c) Purifying the harvest comprising the PCV-2 antigen, preferably the PCV-2 ORF2 antigen by size exclusion chromatography (gel filtration) followed by anion exchange chromatography; and
d) Admixing the purified PCV-2 antigen, preferably the PCV-2 ORF2 antigen with an adjuvant.

According to a preferred embodiment, the size exclusion chromatography and the anion exchange chromatography are performed as described herein, preferably as described in Example 3. Preferably, the two step purification strategy results in an immunogenic composition having purity grade of more than 90% (w/w), preferably more than 95% (w/w) with reference to the total amount of protein included in the immunogenic composition prior to the mixture with the adjuvant. The purity grade can be estimated by Imperial Protein Stain (Pierce) staining after SDS PAGE via NuPAGE 10% Bis-Tris gels (Invitrogen) using the NuPAGE MOPS buffer system (Invitrogen).

As described above, the recovery process of the PCV-2 antigen, preferably the PCV″ ORF2 antigen begins with the separation of cell debris from the expressed PCV-2 ORF2 antigen via a separation step. A preferred separation step includes a micro filtration through a filter having a pore size of about 0.6 μm to about 2 μm, preferably having a pore size of about 0.8 mm to about 1.2 μm.

Thus the immunogenic composition comprising a purified PCV-2 antigen, preferably the PCV-2 ORF2 antigen, and the adjuvant, is obtainable by a process comprising the steps
a) Expressing the PCV-2 antigen, prefer the PCV-2 ORF2 antigen in a host cell;
b) Harvesting the cell culture obtaining PCV-2 antigen, preferably the PCV-2 ORF2 antigen;
c) Filtering the harvest obtained under step b) through a filter having a pore size of 0.6 to 2.0 μm.
d) Purifying the filtrate comprising the PCV-2 antigen, preferably the PCV-2 ORF2 antigen and obtained under step c) by size exclusion chromatography (gel filtration) optionally followed by anion exchange chromatography; and
e) Admixing the purified PCV-2 antigen, prefer the PCV-2 ORF2 antigen with an adjuvant.

According to a preferred embodiment, the micro-filtration, size exclusion chromatography and the anion exchange chromatography are performed as described herein, preferably as described in Example 3. Preferably, the two step purification strategy including the pre-filtration step results in an immunogenic composition having purity grade of more than 90% (w/w), preferably more than 95% (w/w) with reference to the total amount of protein included in the immunogenic composition prior to the mixture with the adjuvant. The purity grade can be estimated by Imperial Protein Stain (Pierce) staining after SDS PAGE via NuPAGE 10% Bis-Tris gels (Invitrogen) using the NuPAGE MOPS buffer system (Invitrogen).

The immunogenic compositions comprising the purified PCV-2 antigen, preferably the purified PCV-2 ORF2 antigen described herein, preferably those obtainable by the methods described herein are characterized by an increased immunogenicity as compared to an immunogenic composition not comprising such a purified PCV-2 antigen or purified PCV-2 ORF2 antigen.

In the event, viral vectors such as a recombinant poxvirus, adenovirus or baculovirus is used to produce the PCV-2 antigen, preferably the PCV-2 ORF2 antigen; it is recommended to inactivate the viral nucleic acid by an appropriate inactivation treatment. Such inactivation may occur anytime during the purification of the PCV-2 antigen, preferably the PCV-2 ORF2 antigen. Thus, inactivation may occur immediately after the harvest of the cell culture fluid comprising the PCV-2 antigen, preferably the PCV-2 ORF2 antigen, or after the micro-filtration of the of PCV-2 antigen, preferably of the PCV-2 ORF2 antigen, if micro-filtration is done, prior or after the purification step, for instance, prior to or after the gel filtration, and prior to or after the anion exchange chromatography, if this is done.

Any conventional inactivation method can be used for purposes of the present aspect of the invention. Thus, inactivation can be performed by chemical and/or physical treatments. In preferred forms, the volume of harvest fluids is determined and the temperature is brought to between about 32° C.-42° C., more preferably between about 34° C.-40° C., and most preferably between about 35° C.-39° C. Preferred inactivation methods include the addition cyclized binary ethylenimine (BEI), preferably in a concentration of about 1 to about 20 mM, preferably of about 2 to about 10 mM, still more preferably of about 2 to about 8 mM, still more preferably of about 3 to about 7 mM, most preferably of about 5 mM. For example the inactivation includes the addition of a solution of 2-bromoethyleneamine hydrobromide (BEA), preferably of about 0.4M, which has been cyclized to 0.2M binary ethylenimine (BEI) in 0.3N NaOH, to the fluids to give a final concentration of about 5 mM BEI. Preferably, the fluids are then stirred continuously for 2-96 hours and the inactivated harvest fluids can be stored frozen at −40° C. or below or between about 1° C.-7° C. After inactivation is completed a sodium thiosulfate solution, preferably at 1.0M is added to neutralize any residual BEI. Preferably, the sodium thiosulfate is added in equivalent amount as compared to the BEI added prior to for inactivation. For example, in the event BEI is added to a final concentration of 5 mM, a 1.0M sodium thiosulfate solution is added to give a final minimum concentration of 5 mM to neutralize any residual BEI.

Prior to the mixing of the purified PCV-2 antigen, preferably of the PCV-2 ORF2 antigen with an adjuvant, it is also recommended to dialyze the purified PCV-2 antigen, preferably the PCV-2 ORF2 antigen against phosphate buffered saline, pH 7.4 or any other physiological buffer.

The methods described above result in an PCV-2 antigen with reduced virucidal activity as defined herein as well as in an improved immunogenicity, if the PCV-2 antigen has a purity grade of more than 50% (w/w), preferably of more than 70% (w/w), even more preferred of more than 80% (w/w), even more preferred of more than 85% (w/w), even more preferred of more than 90% (w/w), most preferred of more than 95% (w/w) with reference to the total amount of protein included in the immunogenic composition prior to the mixture with any adjuvant. However, the purified PCV-2 antigen obtainable according to this method II can also be mixed and used together with an adjuvant, preferably with any of the adjuvants described herein. The preferred adjuvant is a Carbopol, preferably in a concentration of about 0.1 to 10 mg/ml, more preferred in a concentration of 0.5 to 5 mg/ml, most preferably of about 1 mg/ml of the final immunogenic composition.

Again, the present aspect of the invention does not only provide any of the methods described herein, including the alternative method II, it also provides a PCV-2 antigen, preferably a purified PCV-2 antigen, most preferably a purified PCV-2 ORF-2 protein obtainable by any of the methods described herein, including the alternative method II. Moreover, the present invention also provides PCV-2 antigenic compositions comprising a PCV-2 antigen, preferably a purified PCV-2 antigen, most preferably a purified PCV-2 ORF-2 protein obtainable by any of the methods described herein, including the alternative method II. The amount of the PCV-2 antigen, in particular of the purified PCV-2 ORF2 antigen in the final immunogenic composition should be in a range from about 0.25 to about 400 μg per dose with reference to the final immunogenic composition. Preferably the finally immunogenic composition should include an amount of PCV-2 antigen, preferably of PCV-2 ORF2 antigen in a range from about 2 to about 200 μg/dose, even more preferably from about 3 to about 150 μg/dose, still more preferably from about 4 to about 100 μg/dose, still more preferably from about 5 to about 80 μg/dose, still more preferably from about 6 to about 60 μg/dose, even more preferably from about 7 to about 50 μg/dose, even more preferably from about 8 to about 40 μg/dose, still more preferably from about 8 to about 32 μg/dose, even more preferably from about 8 to about 24 μg/dose, and most preferred from about 8 to about 16 μg/dose.

The immunogenic compositions provided herewith, including those obtainable by the method II comprises one or more additional antigens of another disease-causing organism. Those “another disease-causing organisms” are defined above. Preferably the additional antigen is Porcine Reproductive and Respiratory Syndrome Virus. Even more preferably, the Porcine Reproductive and Respiratory Syndrome Virus antigen comprises a live virus, and still more preferably a modified live virus. Still more preferably, the modified live Porcine Reproductive and Respiratory Syndrome Virus antigen comprises a modified live virus strain of ATCC Accession Number VR 2332, and still more preferably comprises INGELVAC® PRRS MLV. In a further aspect of the present application, the additional antigen is Mycoplasma hyopneumoniae. Preferably the Mycoplasma hyopneumoniae antigen is a bacterin, and more preferably, the Mycoplasma hyopneumoniae bacterin is INGELVAC® MYCOFLEX. Most preferred are combinations with, both antigen of Porcine Reproductive and Respiratory Syndrome Virus and Mycoplasma hyopneumoniae.

Due to the increased immunogenicity of the immunogenic composition including the purified PCV-2 antigen, preferably the purified PCV-2 ORF2 antigen provided herewith, the immunogenic compositions can be used for reducing the incidence or reducing the severity of clinical signs caused by or being associated with PCV-2 infections as compared to an animal not receiving that immunogenic composition.

The term “reduction in the incidence of or severity of clinical signs” shall mean that any of such signs are reduced in incidence or severity in animals receiving an administration of the vaccine in comparison with a “control group” of animals when both have been infected with or challenged by the pathogen from which the immunological active component(s) in the vaccine are derived and wherein the control group has not received an administration of the vaccine or immunogenic composition. In this context, the term “decrease” or “reduction” means a reduction of at least 10%, preferably 25%, even more preferably 50%, most preferably of more than 100% in the vaccinated group as compared to the control group not vaccinated.

As used herein, “clinical symptoms” or “clinical signs” shall refer to signs of infection from a pathogen(s) that are directly observable from a live animal such as symptoms. Representative examples will depend on the pathogen selected but can include things such as nasal discharge, lethargy, coughing, elevated fever, weight gain or loss, dehydration, diarrhea, swelling, lameness, and the like. PCV-2 clinical signs can include wasting, paleness of the skin, unthriftiness, respiratory distress, diarrhea, icterus, and jaundice.

Reducing in the incidence of or the severity of clinical signs caused by or being associated with PCV-2 infections in an animal can be reached by the administration of only a single dose of such immunogenic composition to an animal in need of such treatment. However, the immunogenic composition provided herewith can also be administered in two doses or more doses, with an interval of 2 to 4 weeks between the administration of the first dose and the any subsequent dose. Thus, according to a further embodiment the immunogenic composition provided herewith including the purified PCV-2 antigen, preferably the purified PCV-2 ORF2 antigen can be administered in one, two or more doses to an animal in need thereof.

In particular, in a further aspect of the present aspect of the invention, an immunogenic composition comprising a PCV-2 antigenic composition as described above is provided wherein the immunogenic composition, when administered to an animal, reduces lymphoid depletion and inflammation by at least 80% or at least 75% in an animal as compared to an animal not receiving the immunogenic composition. Thus, in a further aspect of the present aspect of the invention, an immunogenic composition is provided comprising a PCV-2 antigenic composition as described above and the immunogenic composition reduces lymphoid depletion and inflammation by at least 80% or at least 75% in an animal that has received an administration of the immunogenic composition as compared to an animal not receiving the immunogenic composition.

In a further aspect of the present aspect of the invention, an immunogenic composition comprising a PCV-2 antigenic composition as described above is provided, wherein the immunogenic composition, when administered to an animal, reduces lung lesions by at least 80% in an animal as compared to an animal not receiving the immunogenic composition. Thus, in a further aspect of the present aspect of the invention, an immunogenic composition comprising a PCV-2 antigenic composition is provided as described above and the immunogenic composition reduces lung lesions by at least 80% in an animal that has received an administration of the immunogenic composition as compared to an animal not receiving the immunogenic composition.

In a further aspect of the present aspect of the invention, an immunogenic composition comprising a PCV-2 antigenic composition, as described above, is provided wherein the immunogenic composition induces a protective immune response against PCV-2 after the administration of one dose of the immunogenic composition. The immunogenic composition comprising a PCV-2 antigenic composition can be of any volume including 1 ml, 2 ml, 3 ml, 4 ml, 5 ml and higher. In preferred forms, 2 ml of the immunogenic composition comprises one dose of the PCV-2 antigen. Thus, in a further aspect of the present aspect of the invention, an immunogenic composition as described above is provided wherein the immunogenic composition comprising a PCV-2 antigenic composition induces a protective immune response against PCV-2 after the administration of one dose of the immunogenic composition. In a further aspect, 2 ml of the immunogenic composition comprises one dose of the PCV-2 antigen.

As used herein, a “protective immune response” refers to a reduced incidence of or reduced severity of clinical, pathological, or histopathological signs or symptoms of infection from a pathogen of interest up to and including the complete prevention of such signs or symptoms.

The term “Pathological” signs shall refer to signs of infection that are observable at the microscopic or molecular level, through biochemical testing, or with the naked eye upon necropsy. For PCV-2, pathological signs will include microscopic and macroscopic lesions on multiple tissues and organs, with lymphoid organs being the most common site for lesions.

The term “Histopathological” signs shall refer to signs of tissue changes resulting from infection.

The terms, “clinical symptoms” or “clinical signs” are defined above.

In a further aspect of the present aspect of the invention, an immunogenic composition comprising a PCV-2 antigenic composition and a PRRRS antigen, preferably any one of the PRRS antigens described herein, as described above, is provided wherein the immunogenic composition induces a protective immune response against PRRS virus after the administration of one dose of the immunogenic composition. Again, any dosage volume can be produced, but in preferred forms, 2 ml of the immunogenic composition comprises one dose of the PRRS antigen and one dose of the PCV-2 antigen. Thus, in a further aspect of the present invention, an immunogenic composition as described above comprising a PRRSV and a PCV-2 antigenic composition as described herein, is provided wherein the immunogenic composition induces a protective immune response against PRRS after the administration of one dose of the immunogenic composition. In a further aspect, 2 ml of the immunogenic composition comprises one dose of the PRRS antigen and one dose of the PCV-2 antigen.

In a further aspect of the present invention, an immunogenic composition comprising a PCV-2 antigenic composition as described herein and Mycoplasma hyopneumoniae antigen as described above, is provided wherein the immunogenic composition induces a protective immune response against Mycoplasma hyopneumoniae after the administration of one dose of the immunogenic composition. Again, any dosage volume can be produced, but in preferred forms, 2 ml of the immunogenic composition comprises one dose of the Mycoplasma hyopneumoniae antigen and one dose of a PCV-2 antigen. Thus, in a further aspect of the present invention, an immunogenic composition as described above is provided wherein the immunogenic composition induces a protective immune response against Mycoplasma hyopneumoniae after the administration of one dose of the immunogenic composition comprising a PCV-2 antigenic composition as described herein and Mycoplasma hyopneumoniae antigen. In a further aspect, 2 ml of the immunogenic composition comprises one dose of the Mycoplasma hyopneumoniae antigen.

In a further aspect of the present aspect of the invention, an immunogenic composition, as described above, is prepared for the administration of 2 ml per dose.

In a further aspect of the present aspect of the invention, a method of reducing one or more clinical symptoms of a PCV-2 infection in an animal as compared to an animal not receiving the immunogenic composition is provided. In general, the method comprises the step of administering to an animal any of the immunogenic compositions comprising a PCV-2 antigenic or composition as described above. Preferably, one or more clinical symptoms of a PCV-2 infection are reduced after the administration of a single dose of the or immunogenic composition. Thus, according to a further aspect of the present aspect of the invention, a method of reducing one or more clinical symptoms of a PCV-2 infection in an animal as compared to an animal not receiving the immunogenic composition comprising a PCV-2 antigenic composition as described herein is provided. In general, the method comprises the step of administering to an animal any of the immunogenic compositions comprising a PCV-2 antigenic composition described above, wherein one or more clinical symptoms of a PCV-2 infection are reduced, preferably after the administration of a single dose of the immunogenic composition comprising a PCV-2 antigenic composition as described herein.

In a further aspect of the present aspect of the invention, a method of reducing one or more clinical symptoms of a PRRS infection, in particular a concurrent infection with (i) PRRS Virus and (ii) a PCV2 of a subtype other than 2a, in an animal as compared to an animal not receiving the immunogenic composition is provided. In general, the method comprises the step of administering to an animal any of the immunogenic compositions described above comprising a PCV-2 antigenic composition as described herein and a PRRS Virus as described herein. Preferably, one or more clinical symptoms of a PRRS infection are reduced after the administration of a single dose of the immunogenic composition comprising a PCV-2 antigenic composition as described herein and a PRRS Virus as described herein. Thus, according to a further aspect of the present aspect of the invention, a method of reducing one or more clinical symptoms of a PRRS infection in an animal as compared to an animal not receiving the immunogenic composition comprising a PCV-2 antigenic composition as described herein and a PRRS Virus as described herein, is provided. Clinical signs of Porcine Reproductive and Respiratory Syndrome Virus (PRRSV) include, but are not limited to inappetance, fever, abortion, transient discoloration, prolonged anoestrus, coughing, respiratory signs, mastitis, agalactia, lethargy, mummified piglets, stillbirths, weak piglets at birth, reduction in farrowing rate, early farrowing, diarrhea, wasting, sneezing, eye discharge, pale skin, mortality, and combinations thereof.

In a further aspect of the present aspect of the invention, a method of reducing one or more clinical symptoms of a Mycoplasma hyopneumoniae infection, in particular a concurrent infection with (i) Mycoplasma hyopneumoniae and (ii) a PCV2 of a subtype other than 2a, in an animal as compared to an animal not receiving the immunogenic composition comprises a PCV-2 antigenic composition as described herein and a Mycoplasma hyopneumoniae antigen as described herein, is provided. In general, the method comprises the step of administering to an animal any of the immunogenic compositions described above. Preferably, one or more clinical symptoms of a Mycoplasma hyopneumoniae infection are reduced after the administration of a single dose of the immunogenic composition comprising a PCV-2 antigenic composition as described herein and a Mycoplasma hyopneumoniae antigen as described herein. Thus, according to a further aspect of the present aspect of the invention, a method of reducing one or more clinical symptoms of a Mycoplasma hyopneumoniae infection, in particular a concurrent infection with (i) Mycoplasma hyopneumoniae and (ii) a PCV2 of a subtype other than 2a, in an animal as compared to an animal not receiving the immunogenic composition comprising a PCV-2 antigenic composition as described herein and a Mycoplasma hyopneumoniae antigen as described herein is provided. Clinical signs of Mycoplasma hyopneumoniae (M. hyo) infection includes but are not limited to, a dry cough, impaired performance, and lung lesions.

The immunogenic composition comprising the purified PCV-2 antigen, preferably the PCV-2 ORF2 antigen as provided herein, has improved immunogenicity. Therefore, the immunogenic composition provided herewith is suitable to improve the immune response in an animal receiving such immunogenic composition. Thus, according to a further embodiment, the present invention provides a method for improving the immune response in an animal against PCV-2 comprising the step: administering a the immunogenic composition as described herein and having a purified PCV-2 antigen, preferably a purified PCV-2 ORF-2 protein as provided herewith, to an animal in need thereof. According to a preferred aspect, the PCV-2 antigen, preferably the PCV-2 ORF2 antigen used in such method is purified to an extent of more than 60% (w/w), preferably more than 60% (w/w), even more preferred to more than 70% (w/w), even more preferred to more than 80% (w/w), even more preferred to more than 90% (w/w), most preferred to more than 95% (w/w) with reference to the total amount of protein included in the immunogenic composition. The purity grade can be estimated by Imperial Protein Stain (Pierce) staining after SDS PAGE via NuPAGE 10% Bis-Tris gels (Invitrogen) using the NuPAGE MOPS buffer system (Invitrogen). The PCV-2, and preferably the PCV-2 ORF2 can be purified using conventional methods well known to a person skilled in the art.

### EXAMPLES

### Example 1

This study was an efficacy evaluation of an experimental Porcine Circovirus Vaccine, Type 2, killed Baculovirus Vector (including recombinant baculovirus expressed PCV2a ORF2 protein) against a virulent PCV2-ORF2b (PCV2b) challenge.

30 cesarean-derived colostrum-deprived (CDCD) piglets at seven weeks of age were used in this study and separated into 2 groups; 1) 15 pigs vaccinated with an experimental Porcine Circovirus Vaccine, Type 2, Killed Baculovirus Vector, and 2) 15 non-vaccinated challenged control pigs. On Day 0, Group 1 PCV2a pigs were administered 1 mL of vaccine intramuscularly (IM) with vaccine containing a relative potency (RP) of 1.5 whereas Group 2, non-vaccinated challenge control pigs did not receive any treatment.

On Day 28, all pigs were challenged with virulent PCV2-ORF2b (PCV2b) 1 mL intranasally (IN) and 1 mL IM with an approximate dosage of 3.0 Log₁₀TCID₅₀/mL of live virus. All pigs received 2.0 mL Keyhole Limpet Hemocyanin emulsified in Incomplete Freunds Adjuvant (KLH/ICFA) IM on Days 25 and 31. Pigs were monitored daily for clinical signs, and blood was drawn for serologic testing periodically. On Day 56 all pigs were necropsied and select tissues were collected and gross pathology observations made.

As a whole, PCV-PCV2a vaccinated animals out-performed the challenge control group in all parameters tested. One out of fifteen, (6.67%) vaccinated animals were viremic on Day 56 compared to fifteen out of fifteen (100%) challenge control animals. Also, only one tonsil tissue (6.67%) from one PCV2-ORF2a vaccinated animal was immunohistochemistry (IHC) positive whereas challenge control pigs results were as follows: Tonsil 67% positive, Iliac lymph node 64% positive, Tracheobronchial lymph node 40% positive, and mesenteric lymph node 73% positive.

The data obtained from this study indicate that a PCV2-ORF2a vaccine cross protects against piglets exposed to a virulent PCV2-ORF2b challenge.

### Example 2

This study evaluated the efficacy of Porcine Circovirus Type 2 ORF2a Prototype Vaccine (including recombinant baculovirus expressed PCV2a ORF2 protein) against a PCV2 ORF2b (PCV2b) challenge when given at three weeks of age.

Forty two healthy CDCD pigs (11 pigs from each of 2 litters and 10 pigs from each of 2 litters) were blocked and housed amongst six pens. Pigs within a pen were equally randomized to 1 of 3 treatment groups: Group 1 (Strict Negative Controls) consisted of 6 pigs and received no treatment, Group 2 (Challenge Controls, n=18) received no treatment, and Group 3 (Experimental PCV2a vaccine serial 420-19aB, n=18). An overview of the treatment groups is provided in Table 1.

TABLE 1

| | No. of | | | Day 11 and | | |
|---|---|---|---|---|---|---|
| Group | Pigs | Treatment | Day 0 | Day 17 | Day 14 | Day 42 |
| 1 | 6 | Strict Neg Cont | n/a | n/a | Necropsy | n/a |
| 2 | 18 | none | n/a | KLH/ICFA | PCV2b | Necropsy |
| | | | | Treatment | challenge | |
| 3 | 18 | PCV2a ORF2 protein + | Vaccinate | KLH/ICFA | PCV2b | Necropsy |
| | | Carbopol | | Treatment | challenge | |

On D0 pigs were 24 days of age and Group 3 pigs were administered a 1 mL dose of vaccine intramuscularly (IM). On D11 and D17, all pigs received a 2.0 mL dose of KLH/ICFA, intramuscularly (IM). One pig was removed from Group 2 before challenge (poor condition). On D14 all pigs were challenged with 5.25 log₁₀TCID₅₀/mL of live virulent PCV2b 1.0 mL IM in the right neck and 1.0 mL intranasally. Pigs were examined daily for overall health. Blood samples were collected on D-4, D14, D21, D28, D33 and D42, and sera were tested for PCV2 viremia by quantitative real time polymerase chain reaction on all days with the exception of Day −4. 18 percent (3/17) of the pigs of the control group died before the end of the study in comparison with only 5.5 percent (1/18 pigs) in the vaccinated group. In the control group, two pigs on D39 and one pig on D40 were moribund or found dead in comparison with only one pig on D27 in the vaccinated group. On D42 all surviving pigs were euthanized; a tonsil sample, a tracheobronchial lymph node, a mesenteric lymph node and an iliac lymph node were collected into formalin. A pathologist scored each tissue as negative or positive (severity score of 1 to 3) for lymphoid depletion, inflammation and colonization (immunohistochemistry).

Based on histological results, the mitigated fraction (and lower confidence interval) for lymphoid depletion was 0.627 (0.4259), for lymphoid inflammation was 0.617 (0.4545) and for lymphoid colonization was 0.686 (0.4375). Thus, it was observed that the mitigation of lymphoid depletion, lymphoid inflammation and lymphoid colonization was significant in the vaccinated pigs, in comparison with the challenge control. Also, analysis of the quantitative rt-PCR viremia data revealed a reduction of viral load in vaccinated pigs in comparison with the challenge control.

What is claimed is:

1. A method for the treatment or prevention of an infection with PCV2 of a subtype other than subtype a, the reduction, prevention or treatment of clinical signs caused by an infection with PCV2 of a subtype other than subtype a, or the prevention or treatment of a disease caused by an infection with PCV2 of a subtype other than subtype a comprising the use of a PCV2 subtype a (PCV2a) ORF2 protein or immunogenic composition comprising PCV2a ORF2 protein.

2. The method of claim 1, wherein the infection with PCV2 of a subtype other than subtype a is an infection with PCV2 subtype b (PCV2b) and/or PCV2 subtype c (PCV2c).

3. The method of claim 1, wherein the infection with PCV2 of a subtype other than subtype a is an infection with PCV2b.

4. The method of claim 1, wherein the infection with PCV2 of a subtype other than subtype a is a concurrent infection with PCV2 of a subtype other than subtype a and PCV2a.

5. The method of claim 1, wherein the infection with PCV2 of a subtype other than subtype a is a concurrent infection with PCV2a and PCV2b.

6. The method of claim 4, wherein said concurrent infection with PCV2a is an infection with a PCV2 comprising a polypeptide that is at least 94% or preferably at least 95% identical to the sequence of SEQ ID NO:1 or comprising a polynucleotide which comprises a sequence encoding a polypeptide that is at least 94% or preferably at least 95% identical to the sequence of SEQ ID NO:1.

7. The method of claim 2, wherein the infection with PCV2b is an infection with a PCV2 comprising a polypeptide that is at least 94% or preferably at least 95% identical to the sequence of SEQ ID NO:2 or comprising a polynucleotide which comprises a sequence encoding a polypeptide that is at least 94% or preferably at least 95% identical to the sequence of SEQ ID NO:2.

8. The method of claim 1, wherein said PCV2a ORF2 protein is a recombinant PCV2a ORF2 protein, preferably a recombinant baculovirus expressed PCV2a ORF2 protein.

9. The method of claim 1, wherein said PCV2a ORF2 protein comprises of consists of a sequence that is at least 94% or preferably at least 95% identical to the sequence of SEQ ID No:1.

10. A method of inducing an immune response against a PCV2 of a subtype other than subtype a or the concurrent induction of an immune response against said PCV2 of a subtype other than subtype a and a PCV2a comprising:
administration of a PCV2a ORF2 protein or immunogenic composition comprising PCV2a ORF2 protein, wherein said animal has clinical signs of disease selected from the group consisting of lymphoid depletion, lymphoid inflammation, lymphoid colonization, positive IHC for PCV2 antigen of lymphoid tissue, viremia, nasal shedding, pyrexia, reduced average daily weight gain, lung inflammation, positive IHC for PCV2 antigen of lung tissue, increased mortality, or said disease is PMWS.

11. A method for the treatment or prevention of an infection with PCV2 of a subtype other than subtype a, for the reduction, prevention or treatment of clinical signs caused by an infection with PCV2 of a subtype other than subtype 2a, or for the treatment or prevention of a disease caused by an infection with PCV2 of a subtype other than subtype 2a, comprising:
administering PCV2a ORF2 protein or an immunogenic composition comprising PCV2a ORF2 protein to an animal, wherein said infection with PCV2 of a subtype other than subtype 2a is preferably said infection with PCV2 of a subtype other than subtype a according to claim 1 and wherein said PCV2a ORF2 protein is preferably said PCV2a ORF2 protein is a recombinant baculovirus expressed PCV2a ORF2 protein.

12. The method of claim 11, wherein the prevention, reduction or treatment of an infection with PCV2 of a subtype other than subtype 2a comprises the induction of an immune response against said PCV2 of a subtype other than subtype 2a or the concurrent induction of an immune response against said PCV2 of a subtype other than subtype a and PCV2a, wherein said clinical signs are selected from the group consisting of lymphoid depletion, lymphoid inflammation, lymphoid colonization, positive IHC for PCV2 antigen of lymphoid tissue, viremia, nasal shedding, pyrexia, reduced average daily weight gain, lung inflammation positive IHC for PCV2 antigen of lung tissue, increased mortality, or said disease is PMWS.

13. The method of claim 10, wherein said PCV2a ORF2 protein or said immunogenic composition is administered only once.

14. The method of claim 1, wherein said PCV2a ORF2 protein or said immunogenic composition is administered to an animal, preferably to a swine, more preferably to a pig, in particular preferably to a piglet or a sow.

15. The method of claim 1, wherein said immunogenic composition comprising PCV2a ORF2 protein has reduced virucidal activity and is prepared by a process comprising the steps in the following order:
a. obtaining a first liquid containing an isolated PCV-2 subtype a (PCV2a) ORF2 antigen;
b. removing at least a portion of the first liquid from the PCV-2a ORF2 antigen by an exchange of the portion of the first liquid against a second liquid, wherein the second liquid is different from the first liquid and is non-virucidal, and the exchange of the portion of the first liquid with the second liquid comprises the steps of i) adding the second liquid to the first liquid; and ii) concentrating the PCV-2a ORF2 antigen by removing a portion of the first and second liquids whereby a PCV-2a antigenic composition having reduced virucidal activity as compared to the isolated PCV-2a ORF2 antigen of the first liquid is produced; and
c. wherein the PCV-2 antigenic composition having reduced virucidal activity of step b) causes a loss of less than 0.7 log TCID50 per ml of a live virus or less than 1 log CFU per ml of a live bacterium when the live virus or live bacterium is mixed with the PCV-2 antigenic composition for 2 or more hours.

16. The method of claim 15, wherein the portion of the first liquid is removed from the isolated PCV-2a ORF2 antigen by a filtration step utilizing a filter.

17. The method of claim 15, wherein concentration step ii) and addition step i) are performed substantially simultaneously.

18. The method of claim 15, wherein concentration step ii) and addition step i) are performed at least two times.

19. The method of claim 16, wherein the filter includes a semi-permeable membrane.

20. The method of claim 19, wherein the semi-permeable membrane has an average pore size that is smaller than the isolated PCV-2a ORF2 antigen and prevents passage of at least 90% of the isolated PCV-2a ORF2 antigen through the semi-permeable membrane pores and holds the isolated PCV-2a ORF2 antigen within the filter.

21. The method of claim 16, wherein the filter has an average pore size that prevents passage of at least 90% of proteins of 50 kDa to 500 kDa in size.

22. The method of claim 15, wherein concentration step ii) concentrates the PCV-2a antigen from 3× to 50× as compared to the first liquid.

23. The method of claim 15, wherein the virucidal activity of the PCV-2a antigenic composition is reduced by at least 10% as compared to the isolated PCV-2a ORF2 antigen of the first liquid.

24. The method of claim 16, wherein the method further comprises harvesting the isolated PCV-2a ORF2 antigen remaining after step ii).

25. The method of claim 24, wherein the method further comprises purifying the harvested isolated PCV-2a ORF2 antigen by a chromatographic procedure.

26. The method of claim 25, wherein the isolated PCV-2a ORF2 antigen is purified to a purity grade of more than 50% (w/w) with reference to the total amount of protein.

27. The method of claim 15, wherein the method further comprises admixing the isolated PCV-2a ORF2 antigen remaining after step ii) with a further component selected from the group consisting of pharmaceutically acceptable carriers, adjuvants, diluents, excipients, and combinations thereof.

28. The method of claim 27, wherein the further component is an adjuvant.

29. The method of claim 28, wherein the adjuvant is a Carbomer.

30. The method of claim 15, wherein the isolated PCV-2 ORF2a antigen comprises open reading frame 2 (ORF-2) protein of PCV-2a.

31. The method of claim 30, wherein the isolated PCV-2a ORF2 antigen comprises virus like particles of the ORF-2a protein.

32. The method of claim 15, wherein the method further comprises combining the PCV-2a antigenic composition with at least one additional antigen.

33. The method of claim 32, wherein the at least one additional antigen is selected from the group consisting of an antigen of: Actinobacillus pleuropneumonia; Adenovirus; Alphavirus preferably Eastern equine encephalomyelitis viruses; Bordetella bronchiseptica; Brachyspira spp., preferably B. hyodyentheriae; B. piosicoli; Brucella suis, preferably biovars 1, 2, and 3; Classical swine fever virus; Clostridium spp., preferably Cl. Difficile; Cl. perfringens types A, B, and C, Cl. Novyi, Cl. septicum, Cl. tetani; Coronavirus, preferably Porcine Respiratory Corona virus and Porcine Epidemic Diarrhea Virus; Eperythrozoonosis suis; Erysipelothrix rhsiopathiae; Escherichia coli; Haemophilus parasuis, preferably subtypes 1, 7 and 14; Hemagglutinating encephalomyelitis virus; Japanese Encephalitis Virus; Lawsonia intracellularis; Leptospira spp., preferably Leptospira australis, Leptospira canicola, Leptospira grippotyphosa, Leptospira icterohaemorrhagicae, Leptospira interrogans, Leptospira pomonas, and Leptospira tarassovi; Mycobacterium spp. preferably M. avium, M. intracellulare, and M. bovis; Mycoplasma hyopneumoniae; Pasteurella multocida; Porcine cytomegalovirus; Porcine Parvovirus; Porcine Reproductive and Respiratory Syndrome Virus; Pseudorabies virus; Rotavirus; Salmonella spp., preferably S. thyhimurium and S. choleraesuis; Staph. hyicus; Staphylococcus spp. preferably Streptococcus spp., preferably Strep. suis; Swine herpes virus; Swine Influenza Virus; Swine pox virus; Swine pox virus; Vesicular stomatitis virus; Virus of vesicular exanthema of swine; Leptospira Hardjo; and Mycoplasma hyosynoviae.

34. The method of claim 33, wherein said at least one additional antigen is an antigen of Mycoplasma hyopneumoniae and/or an antigen of Porcine Reproductive and Respiratory Syndrome Virus.

35. The method of claim 33, wherein said at least one additional antigen is an attenuated Porcine Reproductive and Respiratory Syndrome Virus.

36. The method of claim 33, wherein said at least one additional antigen is a Mycoplasma hyopneumoniae bacterin.
